(19) United States
(12) Patent Application Publication
(10) Pub. No.: US 2014/0235790 A1
(43) Pub. Date: Aug. 21, 2014

(54) CARBOHYDRATE-BASED COMPOSITIONS AND METHODS FOR TARGETED DRUG DELIVERY

(71) Applicants: Patrick Stayton, Seattle, WA (US); Matthew Manganiello, Seattle, WA (US); Anthony Convertine, Seattle, WA (US); Daniel M. Ratner, Seattle, WA (US); Yu-Hua Chow, Mercer Island, WA (US); Eun-Ho Song, Seattle, WA (US); Bilal Ghosn, Houston, TX (US); Lynn Schnapp, Houston, TX (US); Salka Keller, Seattle, WA (US); Debashish Roy, Seattle, WA (US)

(72) Inventors: Patrick Stayton, Seattle, WA (US); Matthew Manganiello, Seattle, WA (US); Anthony Convertine, Seattle, WA (US); Daniel M. Ratner, Seattle, WA (US); Yu-Hua Chow, Mercer Island, WA (US); Eun-Ho Song, Seattle, WA (US); Bilal Ghosn, Houston, TX (US); Lynn Schnapp, Houston, TX (US); Salka Keller, Seattle, WA (US); Debashish Roy, Seattle, WA (US)

(73) Assignee: University of Washington through its Center for Commercialization, Seattle, WA (US)

(21) Appl. No.: 14/125,289

(22) PCT Filed: Jun. 11, 2012
(86) PCT No.: PCT/US2012/041962

§ 371 Date: Apr. 3, 2014

Related U.S. Application Data
(60) Provisional application No. 61/495,922, filed on Jun. 10, 2011.
(60) Provisional application No. 61/495,915, filed on Jun. 10, 2011.
(60) Provisional application No. 61/495,908, filed on Jun. 10, 2011.

(51) Int. Cl. A61K 47/48; A61K 51/06; A61K 49/12; A61K 49/00

(52) U.S. Cl. CPC A61K 47/48176; A61K 49/0054; A61K 51/065; A61K 49/12
USPC 525/54.3; 525/293

(57) ABSTRACT

Provided herein are compositions and methods for intracellular delivery. The compositions are polymer compositions in which the polymer serves as a carrier for therapeutic and/or diagnostic agents. The polymer compositions are effective in targeted delivery of therapeutic and/or diagnostic agents to a cell. The polymer compositions include a targeting moiety that includes carbohydrate groups that effectively target specific cell surface receptors. The polymer compositions also include an agent binding moiety that effectively associates the therapeutic and/or diagnostic agent to be delivered to the cell.

### CROSS-REFERENCES TO RELATED APPLICATIONS

This application claims the benefit of U.S. Provisional Application No. 61/495,908, filed Jun. 10, 2011; U.S. Provisional Application No. 61/495,915, filed Jun. 10, 2011; and U.S. Provisional Application No. 61/495,922, filed Jun. 10, 2011, the disclosures of which are each expressly incorporated herein by reference in their entirety.

### STATEMENT OF GOVERNMENT LICENSE RIGHTS

This invention was made with Government support under grant numbers K24HL068796 and 5 R01 EB 002991, both awarded by the National Institutes of Health. The Government has certain rights in the invention.

### BACKGROUND

There remains considerable opportunity for the improvement of targeted delivery of biologics, small molecule drugs, and diagnostic agents. For example, cancer treatment strategies, and cancer vaccination, or immunotherapy, which employs the body's own immune system destroy diseased cells, can benefit from more precisely targeted delivery of biologics or small molecules. However, developing delivery carriers that recognize cell-specific ligands for targeted delivery, while remaining inert to non-specific and non-target cell types, has remained a challenge.

Immunotherapy provides the potential for increased efficacy over conventional therapeutic methods, such as for cancer or infectious diseases. Immunotherapy can reduce the dose of toxic chemotherapeutic agents and radiation measures that are often accompanied by devastating side effects due, in part, to their lack of specificity. Immunotherapy is especially promising in the prevention of the metastatic spreading of cancerous cells, which remains a significant threat even after the successful removal of primary tumor tissue by standard methods. Improving the efficacy and applicability of immunotherapy techniques could therefore lead to improved prognosis and quality of life for cancer patients undergoing treatment.

Furthermore, modulation of the immune responses through targeted immunotherapy can ameliorate conditions associated with overactive, or inappropriate endogenous immune responses. For example, overexuberant inflammatory responses to injury or infection can result in collateral tissue damage, prolonged discomfort, and impaired wound healing. Additionally, excessive M2 macrophage responses can result in tissue fibrosis. Other conditions associated with dysregulated macrophage activity include chronic ulcers, allergic asthma, atherosclerosis, various autoimmune disorders, and fibrotic diseases.

Despite the advances in targeted drug delivery, there remains a need for an effective, delivery system for therapeutic agents and/or diagnostic agents that is simple to produce and applicable to a wide variety of agents. The present invention seeks to fulfill this need and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the invention provides polymeric compositions useful for targeted intracellular delivery. In one embodiment, the polymer composition is a block copolymer having a first block comprising repeating units having pendant carbohydrate groups and a second block comprising repeating units having pendant functional groups suitable for associating a therapeutic agent and/or a diagnostic agent to the block copolymer. In this embodiment, the diblock copolymer includes a first block that is a cell targeting block in which the repeating units have pendant carbohydrate groups and a second block that is the therapeutic agent and/or diagnostic agent binding/carrier/delivery block.

In another aspect, the invention provides a nanoparticle. The nanoparticle comprises a plurality of copolymers of the invention that include repeating units having membrane destabilizing functionality.

In another aspect, the invention provides pharmaceutical compositions. The pharmaceutical composition comprises a pharmaceutically acceptable carrier and at least one polymer compositions, copolymers or nanoparticle of the invention.

In another aspect, the invention provides a method for introducing one or more of a therapeutic and/or diagnostic agent into a cell, comprising contacting a cell with at least one copolymer as described herein.

In another aspect, a method of modifying the level of expression of an endogenous protein is provided. In some embodiments, the method comprises silencing or reducing the level of expression of the endogenous protein.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 schematically illustrates an embodiment of RAFT-mediated synthesis of a copolymer of the present invention comprising pendant carbohydrate targeting groups and pendant functional groups, subsequent fluorophore conjugation via a pendant functional group, and a proposed mechanism of MRC-1-mediated macrophage uptake of the copolymers.

FIG. 2 graphically illustrates the time-dependent agglutination of ConA mediated by copolymers of the present invention. Copolymer p(ManEMA) is indicated as “Man”, p(GalEMA) is indicated as “Gal”, and p(GlcNAcEMA) is indicated as “GlcNAc”. Agglutination was assayed with UV-Vis spectroscopy, where ConA was at 1 μM and the copolymers are at 10 μM (based on number of carbohydrate repeats).

FIG. 3 graphically illustrates the dose dependent internalization of the AF488-labeled copolymers of the present invention as determined by flow cytometry. BMDMs were incubated with the AF488-labeled copolymers for 15 min at 37° C. The copolymers are identified on the X-axis in reference to the pendant carbohydrate groups. Data are reported as mean fluorescence intensity±standard deviation from three independent experiments.

FIG. 4 graphically illustrates the competitive uptake of copolymers of the present invention by BMDMs. BMDMs were preincubated with 15 μM unlabeled copolymers for 15 min followed by 1 μM AF488-labeled copolymer for 30 min at 37° C. The unlabeled copolymer used for competition is indicated for each bar. “NC” represents no competition. The AF488-labeled copolymers are indicated for each horizontal bar below the x-axis in reference to the pendant carbohydrate groups. Data are reported as mean fluorescence intensity±standard deviation as determined by flow cytometry from three independent experiments.

FIG. 5 graphically illustrates the uptake of the mannose copolymer comprising pendant mannose groups (i.e., p(ManEMA)) over time in different macrophage types. AF488-labeled pManEMA (1 μM) uptake by unstimulated (M0), LPS-stimulated (M1), or IL-4/IL-13 stimulated (M2) BMDM's is illustrated as mean fluorescence intensity as determined by flow cytometry. Standard deviation is indicated with error bars (n=3).

FIGS. 6A and B graphically illustrates the in vitro cell specific internalization of AF488-labeled copolymers of the present invention (1.5 μM) by BMDMs and (A) primary mouse lung fibroblasts (MLF), or (B) mouse lung epithelial cells (MLE-12), after 2 h (A) or 24 h (B) incubation.

FIG. 7 graphically illustrates the internalization of copolymers of the present invention by alveolar macrophages following intratracheal administration in vivo. Mice were intratracheally administered 10 μM of the indicated AF488-labeled copolymers, followed by bronchoalveolar lavage (BAL) after 30 minutes. BAL cells were analyzed by flow cytometry for fluorescence. Data are reported as mean fluorescence intensity±standard deviation from three independent experiments. * indicates p<0.01 as compared to no polymer.

FIG. 8 graphically illustrates the particle size measurement of micellic assemblies comprising triblock copolymers of the present invention complexed with pDNA. The particle size is illustrated as a function of charge ratio (+/−) as determined by dynamic light scattering (DLS). Mean diameter was determined from the lognormal size distribution. Data are compiled from one experiment with each sample run in triplicate. Error bars represent the standard deviation as calculated from the polydispersity index (PDI) of the particles.

FIG. 9 graphically illustrates the particle size measurements of micellic assemblies comprising triblock copolymers of the present invention. The particle size is illustrated as a function of pH as determined by DLS. All measurements were performed at a copolymer concentration of 1 mg/mL in 100 mM sodium phosphate buffer with 150 mM NaCl. Diameter values were determined from the lognormal number average. Error bars represent the standard deviation as calculated from the polydispersity index (PDI) of the particles.

FIG. 10 graphically illustrates the hemolytic activity of the triblock copolymers of the present invention as a function of pH. All measurements were performed at a copolymer concentration of 20 μg/mL. The extent of hemolysis was determined as a function of free hemoglobin detected at 492 nm. Hemolytic activity was normalized relative to a positive control, 1% v/v Triton X-100, and the data represent single experiment conducted in triplicate±standard deviation.

FIG. 11 graphically illustrates the time-dependent, copolymer-mediated agglutination of Concanvalin A by triblock with a block comprising pendant mannose groups, and related diblock copolymer with a block comprising the functional groups and the pH-responsive micelle forming block, as measured via UV-Vis spectroscopy. The spiked sample was treated with α-D-mannose (final concentration of 20 mM) 10 minutes after the triblock copolymer and lectin were mixed together.

FIG. 12 graphically illustrates the in vitro uptake in BMDCs of Alexa488-labeled linear copolymers of the present invention, comprising the indicated pendant carbohydrates and functional groups, after 4 hours treatment at 1.5 μM. Samples designated with a “+” were co-incubated with the unlabeled copolymer with pendant mannose groups at a concentration of 15 μM. Error bars represent the standard deviation of samples run in triplicate±standard deviation.

### DETAILED DESCRIPTION

The present invention provides compositions and methods for intracellular delivery. The compositions are polymer compositions in which the polymer serves as a carrier for therapeutic and/or diagnostic agents. The polymer compositions are effective in targeted delivery of therapeutic and/or diagnostic agents to a cell. The polymer compositions include a targeting moiety that includes carbohydrate groups that effectively target specific cell surface receptors. The polymer compositions also include an agent binding moiety that effectively associates the therapeutic and/or diagnostic agent to be delivered to the cell. The polymer compositions optionally include an endosomal delivery moiety that provides for transport of the composition in vivo and renders the composition membrane destabilizing once the composition has been introduced into the target cell. The methods of the invention utilize the compositions of the invention for target delivery of one or more therapeutic and/or diagnostic agents to a cell.

In accordance with the foregoing, in one aspect, the invention provides polymeric compositions useful for targeted intracellular delivery.

In a first embodiment, the polymer composition is a block copolymer having a first block comprising repeating units having pendant carbohydrate groups and a second block comprising repeating units having pendant functional groups suitable for associating a therapeutic agent and/or a diagnostic agent to the block copolymer. In this embodiment, the diblock copolymer includes a first block that is a cell targeting block in which the repeating units have pendant carbohydrate groups and a second block that is the therapeutic agent and/or diagnostic agent binding/carrier/delivery block.

As used herein, the term “copolymer” refers to a polymer that is the result of polymerization of two or more different monomers. A “block” copolymer refers to a structure comprising one or a combination of constitutional or monomeric units, used interchangeably herein. Such constitutional or monomeric units can comprise residues of polymerized monomers. In some embodiments, a block copolymer described herein comprises non-lipidic constitutional or monomeric units. In some embodiments, the block copolymer comprises a plurality of blocks, such as two, three, four or more. For example, in some embodiments, the block copolymer is a diblock copolymer, which comprises two blocks. A schematic generalization of a diblock polymer is represented by the following: [AₐB_{b}C_{c} . . . ]ₘ-[XₓY_{y}Z_{z} . . . ]ₙ, although it will be understood that the exemplary schematic can be expanded to represent a copolymer with three (i.e., triblock) or more blocks. In this exemplary scheme, each letter stands for a constitutional or monomeric unit, and wherein each subscript to a constitutional unit represents the mole fraction of that unit in the particular block, the ellipses indicate that there may be more (or there may also be fewer) constitutional units in each block and m and n indicate the molecular weight of each block in the diblock copolymer. As suggested by the schematic, in some instances, the number and the nature of each constitutional unit is separately controlled for each block. The schematic is not meant and should not be construed to infer any relationship whatsoever between the number of constitutional units or the number of different types of constitutional units in each of the blocks. Nor is the schematic meant to describe any particular number or arrangement of the constitutional units within a particular block. In each block the constitutional units may be disposed in a purely random, an alternating random, a regular alternating, a regular block or a random block configuration unless expressly stated to be otherwise.

As used herein, the brackets enclosing the constitutional units are not meant and are not to be construed to mean that the constitutional units themselves form blocks. That is, the constitutional units within the square brackets may combine in any manner with the other constitutional units within the block, i.e., purely random, alternating random, regular alternating, regular block or random block configurations. The block copolymers described herein are, optionally, alternate, gradient or random block copolymers. In some embodiments' the block copolymers are dendrimer, star or graft copolymers.

In one embodiment, the first block further comprises repeating units having neutral pendant groups. In another embodiment, the second block further comprises repeating units having neutral pendant groups. In a further embodiment, the first and second blocks further comprise repeating units having neutral pendant groups.

As used herein, “neutral pendant groups” refers to pendant groups characterized a lack of charge. In some embodiments, the neutral pendant groups is/are hydrophilic. The inclusion of neutral pendant groups facilitates the synthesis of the copolymers of the present invention by circumventing requirements for expensive or difficult to synthesize carbohydrate monomers, and/or highly reactive carbohydrates to achieve the desired targeting effect. Without being bound by any particular theory, the inclusion of neutral pendant groups allows the sugar monomer containing material to be dissolved in a larger range of solvents at the various stages of copolymer synthesis, thus allowing some of the more complex polymer architectures (e.g. di- and triblock copolymers, as described herein) to be prepared and characterized. This is related to the fact that polymers comprising deprotected sugars monomers have limited solubility in non-aqueous solvents, thus presenting a significant technical barrier to the preparation of block copolymers with additional segments that are not soluble in water. Moreover, for most or all medical applications the copolymers of the present invention must be characterized for the molecular weight and heterogeneity (polydispersity). Such characterization is challenging without the addition of a neutral hydrophilic groups because of the differing solubility of the individual block segments. The addition of a neutral, hydrophilic group changes the solubility of the parent polymer allowing common characterization techniques, such as gel permeation chromatography (GPC) and nuclear magnetic resonance spectroscopy (NMR), to be conducted in a common solvent for all block copolymer segments. In some embodiments, a hydrophilic neutral group is to achieve maximum targeting specificity the sugar when in the aqueous phase.

Neutral moieties useful for inclusion in the copolymers of the present invention are known in the art. Exemplary neutral hydrophilic comonomers include 2-hydroxypropyl methacrylamide (HPMA) and polyethylene glocyol monomethacrylate (PEGMA). A person of ordinary skill in the art will understand that the mole fraction of the neutral pendant group in the copolymer, or copolymer block, can vary widely based on the nature of the carbohydrate pendant group, the neutral pendant group, and the solvents used. For example, HPMA can facilitate efficient copolymerization of acrylamide-based nonprotected carbohydrate monomers under aqueous conditions. For this type of system it might be preferable to use a relatively high mole fraction of HPMA, such as 0.5, relative to the carbohydrate group in order to achieve the desired solubility properties for the resultant copolymer. In the context of the embodiment described below in formula (I), a mole fraction of 0.5 between HPMA and the carbohydrate group (in the first block) would result in “a” and “b” each having a mole fraction of 0.5.

For larger hydrophilic comonomers, such as polyethylene glycol monomethacrylate (PEGMA), it is possible to use a much smaller mole ratio because the neutral group can be 10-50 times bigger than the carbohydrate group. While presenting a different overall copolymer architecture, large neutral groups can confer advantages in terms of material cost and ease of synthesis/use. For example PEGMA systems (320, 950, and 2000 g/mol) are commercially available for a methacrylate-based carbohydrate monomers in either the protected or deprotected form. In such a system, the PEGMA comonomer (if sufficiently large) can alter the solubility and intrinsic material properties of the copolymer (or copolymer block) at mole ratios as low as 0.1. In the context of the embodiment described below in formula (I), a mole fraction of 0.1 between PEGMA and the carbohydrate group (in the first block) would result in “a” being 0.9 and “b” being 0.1. There is an important distinction between adding the neutral non-carbohydrate component as a separate block or as a diluent (copolymer) within the carbohydrate block. The range of realistically achievable sizes and compositions will be limited for a discrete neutral block while the diluent approach has broader scope.

Thus, as described below in the embodiment of formula (I), the mole fraction of the repeating unit comprising the pendant neutral group within the first block (see “b”) can range from 0.0 to 0.99.

In a specific embodiment, the invention provides a copolymer having formula (I):

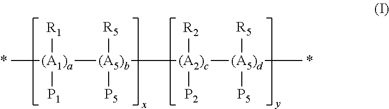

wherein

A₁(R₁)(P₁) is a first block repeating unit, wherein A₁ is a backbone of the repeating unit for the first block, R₁ is a substituent of the repeating unit selected from the group consisting of hydrogen and C1-C8 alkyl optionally substituted with one or more fluorine atoms, and P₁ is a pendant group comprising a carbohydrate,

A₂(R₂)(P₂) is a second block repeating unit, wherein A₂ is a backbone of the repeating unit for the second block, R₂ is a substituent of the repeating unit selected from the group consisting of hydrogen and C1-C8 alkyl optionally substituted with one or more fluorine atoms, and P₂ is a pendant group comprising a functional group suitable for associating a therapeutic or diagnostic agent to the block copolymer, and

A₅(R₅)(P₅) is a repeating unit, wherein A₅ is a backbone of the repeating unit, R₅ is a substituent of the repeating unit selected from the group consisting of hydrogen and C1-C8 alkyl optionally substituted with one or more fluorine atoms, and P₅ is a pendant group comprising a neutral group.

In some embodiments of the copolymer having formula (I), a is the mole fraction of the repeating unit in the first block, wherein a is from 0.01 to 1.0. In some embodiments, a is from 0.01 to 0.5. In some embodiments, a is from 0.1 to 0.4. In some embodiments, a is from 0.2 to 0.3. In some embodiments, a is from 0.2 to 0.8. In some embodiments, a is from 0.3 to 0.7. In some embodiments, a is from 0.4 to 0.6. In some embodiments, a is from 0.5 to 1.0. In some embodiments, a is from 0.6 to 0.9. In some embodiments, a is from 0.7 to 0.8.

In some embodiments of the copolymer having formula (I), b is the mole fraction of the repeating unit in the first block, wherein b is from 0.0 to 0.99. In some embodiments, b is from 0.0 to 0.5. In some embodiments, b is from 0.1 to 0.4. In some embodiments, b is from 0.2 to 0.3. In some embodiments, b is from 0.2 to 0.8. In some embodiments, b is from 0.3 to 0.7. In some embodiments, b is from 0.4 to 0.6. In some embodiments, b is from 0.5 to 0.99. In some embodiments, b is from 0.6 to 0.9. In some embodiments, b is from 0.7 to 0.8.

In some embodiments of the copolymer having formula (I), c is the mole fraction of the repeating unit in the second block, wherein c from 0.01 to 1.0. In some embodiments, c is from 0.01 to 0.5. In some embodiments, c is from 0.1 to 0.4. In some embodiments, c is from 0.2 to 0.3. In some embodiments, c is from 0.2 to 0.8. In some embodiments, c is from 0.3 to 0.7. In some embodiments, c is from 0.4 to 0.6. In some embodiments, c is from 0.5 to 1.0. In some embodiments, c is from 0.6 to 0.9. In some embodiments, c is from 0.7 to 0.8.

In some embodiments of the copolymer having formula (I), d is the mole fraction of the repeating unit in the second block, wherein d is from 0.0 to 0.99. In some embodiments, d is from 0.0 to 0.5. In some embodiments, d is from 0.1 to 0.4. In some embodiments, d is from 0.2 to 0.3. In some embodiments, d is from 0.2 to 0.8. In some embodiments, d is from 0.3 to 0.7. In some embodiments, d is from 0.4 to 0.6. In some embodiments, d is from 0.5 to 0.99. In some embodiments, d is from 0.6 to 0.9. In some embodiments, d is from 0.7 to 0.8.

In some embodiments of the copolymer having formula (I), x is the mole fraction of the first block, wherein x is from 0.01 to about 0.99. In some embodiments, x is from 0.01 to 0.5. In some embodiments, x is from 0.1 to 0.4. In some embodiments, x is from 0.2 to 0.3. In some embodiments, x is from 0.2 to 0.8. In some embodiments, x is from 0.3 to 0.7. In some embodiments, x is from 0.4 to 0.6. In some embodiments, x from 0.5 to 0.99. In some embodiments, x is from 0.6 to 0.9. In some embodiments, x is from 0.7 to 0.8. In some embodiments, x is about 0.5. As used herein, the term “about” refers to a slight permissible variation of 10% around the reference number. For example, “about 0.5” refers to a range from 0.45 to 0.55, and “about 0.33” refers to a range from 0.3 to 0.36. In some embodiments, x is 0.5.

In some embodiments of the copolymer having formula (I), y is the mole fraction of the second block, wherein y is from 0.01 to about 0.99. In some embodiments, y is from 0.01 to 0.5. In some embodiments, y is from 0.1 to 0.4. In some embodiments, y is from 0.2 to 0.3. In some embodiments, y is from 0.2 to 0.8. In some embodiments, y is from 0.3 to 0.7. In some embodiments, y is from 0.4 to 0.6. In some embodiments, y is from 0.5 to 0.99. In some embodiments, y is from 0.6 to 0.9. In some embodiments, y is from 0.7 to 0.8. In some embodiments, y is about 0.5. In some embodiments, y is 0.5.

It will be appreciated that the present invention includes embodiments of any combination of the ranges and subranges described herein for each of the variables of formula (I).

As used herein, the term “backbone of the repeating unit” refers to a residue that is derived from suitable polymerizable monomer. In some embodiments, the backbone of the block copolymer is non-peptidic and/or nonlipidic.

In a second embodiment, the polymer composition is a block copolymer of the first embodiment that further comprises a third block coupled to the terminus of the second block, the third block comprising repeating units having membrane destabilizing functionality. While any membrane destabilizing pendant groups can be used in the embodiments provided herein, in one embodiment the membrane destabilizing functionality is provided by pendant carboxylic acid groups. In another embodiment, the membrane destabilizing functionality is provided by pendant amine groups. Neutral pendant groups may also be incorporated with the carboxylic acid and/or amine groups in the third block. In this embodiment, the polymer composition is a triblock copolymer. The third block is a pH-responsive block effective for endosomal release and micelle formation. The third block is a membrane destabilizing block once the composition has been introduced into the target cell. In some embodiments, the triblock copolymer is endosomal membrane disruptive at about pH 5 or below (hydrophobic and unimeric) and hydrophobic at physiological pH (about pH 7) (nanoparticle and micelle forming).

As used herein, the term “membrane destabilizing” refers to a polymer or composition that directly or indirectly elicit a change (e.g., a permeability change) in a cellular membrane structure (e.g., an endosomal membrane) so as to permit an agent (e.g., therapeutic and/or diagnostic agent, e.g., a protein or peptide, oligonucleotide, or detectable agent) to pass through such membrane structure, for example, to enter a cell or to exit a cellular vesicle (e.g., an endosome). A membrane destabilizing polymer can be (but is not necessarily) a “membrane disruptive” polymer. A membrane disruptive polymer can directly or indirectly elicit lysis of a cellular vesicle or otherwise disrupt a cellular membrane (e.g., as observed for a substantial fraction of a population of cellular membranes). Generally, membrane destabilizing or membrane disruptive properties of polymers can be assessed by various means. In one non-limiting approach, a change in a cellular membrane structure can be observed by assessment in assays that measure (directly or indirectly) release of an agent (e.g., protein or peptide) from cellular membranes (e.g., endosomal membranes), for example, by determining the presence or absence of such agent, or an activity of such agent, in an environment external to such membrane. Another non-limiting approach involves measuring red blood cell lysis (hemolysis), e.g., as a surrogate assay for a cellular membrane of interest. Such assays may be done at a single pH value or over a range of pH values, as described below in the context of an illustrative embodiment of the present disclosure.

The terms “endosomal membrane disruptive” and “endosomolytic” refer to a polymer or composition having the effect of increasing the permeability of the endosomal membrane of an endosome.

As used herein, the terms “pH-responsive, membrane-destabilizing” or “pH-dependent, membrane-destabilizing” refer to a polymer or composition that is at least partially, predominantly, or substantially hydrophobic and is membrane destabilizing in a pH dependent manner. In certain instances, a pH-responsive membrane destabilizing polymer block is a hydrophobic polymeric segment of a copolymer and/or comprises a plurality of hydrophobic species; and comprises a plurality of chargeable species. In some embodiments, the chargeable species is anionic. In some embodiments, the anionic chargeable species is anionic at about neutral pH. In further or alternative embodiments, the anionic chargeable species is non-charged at a lower, e.g., endosomal pH. In some embodiments, the membrane destabilizing chargeable hydrophobe comprises a plurality of cationic species. The pH dependent membrane-destabilizing chargeable hydrophobe comprises a non-peptidic and non-lipidic polymer backbone. For example, a pH-responsive, membrane-destabilizing block may possess anionic repeat units the substituents of which are predominantly ionized (anions) at one pH, e.g., pH 7.4, and predominantly neutral at a lesser pH, e.g., pH 5.0 whereby the pH-responsive, membrane-destabilizing group or block becomes increasingly hydrophobic as a function of the drop in pH from 7.4 to 5.0.

In some embodiments, the copolymers of the invention comprising a pH-responsive block are membrane-destabilizing at a pH of about 6.5 or lower, preferably at a pH ranging from about 5.0 to about 6.5, or at a pH of about 6.2 or lower, preferably at a pH ranging from about 5.0 to about 6.2, or at a pH of about 6.0 or lower, preferably at a pH ranging from about 5.0 to about 6.0. For example, in one embodiment, the polymer is membrane-destabilizing at a pH of or less than about 6.2, of or less than about 6.5, of or less than about 6.8, of or less than about 7.0. In certain embodiments, membrane destabilization is of any cellular membrane such as, for example, an extracellular membrane, an intracellular membrane, a vesicle, an organelle, an endosome, a liposome, or a red blood cell. In some embodiments, polymeric carriers are membrane destabilizing (e.g., in an aqueous medium) at an endosomal pH.

In one embodiment, the first block of the triblock copolymer further comprises repeating units having neutral pendant groups. In another embodiment, the second block further comprises repeating units having neutral pendant groups. In a further embodiment, the third block further comprises repeating units having neutral pendant groups. In other embodiments, the first and second, the first and third, the second and third, or the first, second, and third blocks further comprise repeating units having neutral pendant groups.

In a specific embodiment, the invention provides a copolymer having formula (II):

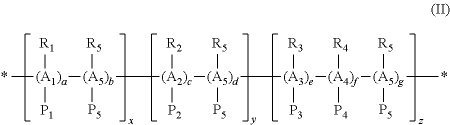

wherein

A₁(R₁)(P₁) is a first block repeating unit, wherein A₁ is a backbone of the repeating unit for the first block, R₁ is a substituent of the repeating unit selected from the group consisting of hydrogen and C1-C8 alkyl optionally substituted with one or more fluorine atoms, and P₁ is a pendant group comprising a carbohydrate,

A₂(R₂)(P₂) is a second block repeating unit, A₂ is a backbone of the repeating unit for the second block, R₂ is a substituent of the repeating unit selected from the group consisting of hydrogen and C1-C8 alkyl optionally substituted with one or more fluorine atoms, and P₂ is a pendant group comprising a functional group suitable for associating a therapeutic or diagnostic agent to the block copolymer,

A₃(R₃)(P₃) is a third block repeating unit, wherein A₃ is a backbone of the repeating unit for the third block, R₃ is a substituent of the repeating unit selected from the group consisting of hydrogen and C1-C8 alkyl optionally substituted with one or more fluorine atoms, and P₃ is a pendant group comprising a carboxylic acid,

A₄(R₄)(P₄) is a third block repeating unit, wherein A₄ is a backbone of the repeating unit for the third block, R₄ is a substituent of the repeating unit selected from the group consisting of hydrogen and C1-C8 alkyl optionally substituted with one or more fluorine atoms, and P₄ is a pendant group comprising an amine, and

A₅(R₅)(P₅) is a repeating unit, wherein A₅ is a backbone of the repeating unit for the repeating unit, R₅ a is a substituent of the repeating unit selected from the group consisting of hydrogen and C1-C8 alkyl optionally substituted with one or more fluorine atoms, and P₅ is a pendant group comprising a neutral group.

In some embodiments of the copolymer having formula (II), a is the mole fraction of the repeating unit in the first block, wherein a is from 0.01 to 1.0. In some embodiments, a is from 0.01 to 0.5. In some embodiments, a is from 0.1 to 0.4. In some embodiments, a is from 0.2 to 0.3. In some embodiments, a is from 0.2 to 0.8. In some embodiments, a is from 0.3 to 0.7. In some embodiments, a is from 0.4 to 0.6. In some embodiments, a is from 0.5 to 1.0. In some embodiments, a is from 0.6 to 0.9. In some embodiments, a is from 0.7 to 0.8.

In some embodiments of the copolymer having formula (II), b is the mole fraction of the repeating unit in the first block, wherein b is from 0.0 to 0.99. In some embodiments, b is from 0.0 to 0.5. In some embodiments, b is from 0.1 to 0.4. In some embodiments, b is from 0.2 to 0.3. In some embodiments, b is from 0.2 to 0.8. In some embodiments, b is from 0.3 to 0.7. In some embodiments, b is from 0.4 to 0.6. In some embodiments, b is from 0.5 to 0.99. In some embodiments, b is from 0.6 to 0.9. In some embodiments, b is from 0.7 to 0.8.

In some embodiments of the copolymer having formula (II), c is the mole fraction of the repeating unit in the second block, wherein c from 0.01 to 1.0. In some embodiments, c is from 0.01 to 0.5. In some embodiments, c is from 0.1 to 0.4. In some embodiments, c is from 0.2 to 0.3. In some embodiments, c is from 0.2 to 0.8. In some embodiments, c is from 0.3 to 0.7. In some embodiments, c is from 0.4 to 0.6. In some embodiments, c is from 0.5 to 1.0. In some embodiments, c is from 0.6 to 0.9. In some embodiments, c is from 0.7 to 0.8.

In some embodiments of the copolymer having formula (II), d is the mole fraction of the repeating unit in the second block, wherein d is from 0.0 to 0.99. In some embodiments, d is from 0.0 to 0.5. In some embodiments, d is from 0.1 to 0.4. In some embodiments, d is from 0.2 to 0.3. In some embodiments, d is from 0.2 to 0.8. In some embodiments, d is from 0.3 to 0.7. In some embodiments, d is from 0.4 to 0.6. In some embodiments, d is from 0.5 to 0.99. In some embodiments, d is from 0.6 to 0.9. In some embodiments, d is from 0.7 to 0.8.

In some embodiments of the copolymer having formula (II), e is the mole fraction of the repeating unit in the third block, wherein e is from 0.0 to 1.0. In some embodiments, e is from 0.01 to 0.5. In some embodiments, e is from 0.1 to 0.4. In some embodiments, e is from 0.2 to 0.3. In some embodiments, e is from 0.2 to 0.8. In some embodiments, e is from 0.3 to 0.7. In some embodiments, e is from 0.4 to 0.6. In some embodiments, e is from 0.5 to 1.0. In some embodiments, e is from 0.6 to 0.9. In some embodiments, e is from 0.7 to 0.8.

In some embodiments of the copolymer having formula (II), f is the mole fraction of the repeating unit in the third block, wherein f is from 0.0 to 1.0, 1.0, wherein e+f is at least 0.01. In some embodiments, f is from 0.01 to 0.5. In some embodiments, f is from 0.0 to 0.4. In some embodiments, f is from 0.2 to 0.3. In some embodiments, f is from 0.2 to 0.8. In some embodiments, f is from 0.3 to 0.7. In some embodiments, f is from 0.4 to 0.6. In some embodiments, f is from 0.5 to 0.99. In some embodiments, f is from 0.6 to 0.9. In some embodiments, f is from 0.7 to 0.8.

In some embodiments of the copolymer having formula (II), g is the mole fraction of the repeating unit in the third block, wherein g is from 0.0 to 0.99. In some embodiments, g is from 0.0 to 0.5. In some embodiments, g is from 0.1 to 0.4. In some embodiments, g is from 0.2 to 0.3. In some embodiments, g is from 0.2 to 0.8. In some embodiments, g is from 0.3 to 0.7. In some embodiments, g is from 0.4 to 0.6. In some embodiments, g is from 0.5 to 0.99. In some embodiments, g is from 0.6 to 0.9. In some embodiments, g is from 0.7 to 0.8.

In some embodiments of the copolymer having formula (II), x is the mole fraction of the first block, wherein x is from 0.01 to about 0.98. In some embodiments, x is from 0.01 to 0.5. In some embodiments, x is from 0.1 to 0.4. In some embodiments, x is from 0.2 to 0.3. In some embodiments, x is from 0.2 to 0.8. In some embodiments, x is from 0.3 to 0.7. In some embodiments, x is from 0.4 to 0.6. In some embodiments, x is from 0.5 to 0.98. In some embodiments, x is from 0.6 to 0.9. In some embodiments, x is from 0.7 to 0.8. In some embodiments, x is about 0.33. In some embodiments, x is 0.33

In some embodiments of the copolymer having formula (II), y is the mole fraction of the second block, wherein y is from 0.01 to about 0.98. In some embodiments, y is from 0.01 to 0.5. In some embodiments, y is from 0.1 to 0.4. In some embodiments, y is from 0.2 to 0.3. In some embodiments, y is from 0.2 to 0.8. In some embodiments, y is from 0.3 to 0.7. In some embodiments, y is from 0.4 to 0.6. In some embodiments, y is from 0.5 to 0.98. In some embodiments, y is from 0.6 to 0.9. In some embodiments, y is from 0.7 to 0.8. In some embodiments, y is about 0.33. In some embodiments, y is 0.33.

In some embodiments of the copolymer having formula (II), z is the mole fraction of the third block, wherein z is from 0.01 to about 0.98. In some embodiments, z is from 0.01 to 0.5. In some embodiments, z is from 0.1 to 0.4. In some embodiments, z is from 0.2 to 0.3. In some embodiments, z is from 0.2 to 0.8. In some embodiments, z is from 0.3 to 0.7. In some embodiments, z is from 0.4 to 0.6. In some embodiments, z is from 0.5 to 0.98. In some embodiments, z is from 0.6 to 0.9. In some embodiments, z is from 0.7 to 0.8. In some embodiments, z is about 0.33. In some embodiments, z is 0.33.

It will be appreciated that the present invention includes embodiments of any combination of the ranges and subranges described herein for each of the variables of formula (II).

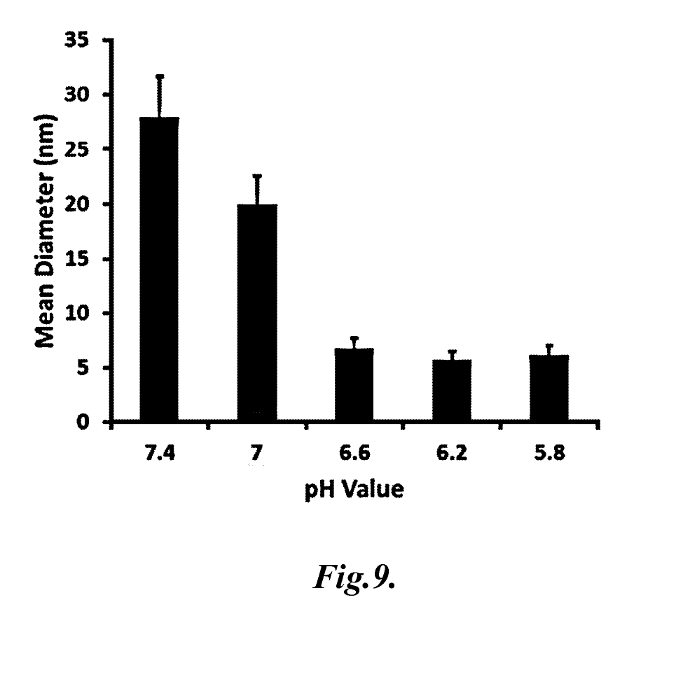

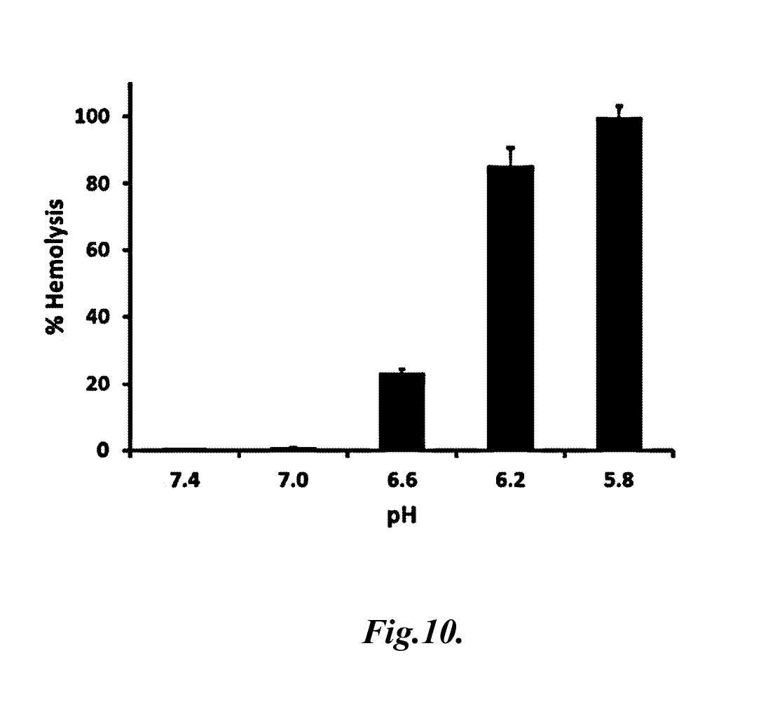

The synthesis of an exemplary triblock copolymer is described below in the representative embodiment entitled “Evaluation and Characterization of Carbohydrate-Containing Copolymer-Mediated Targeting for Copolymer Micelles Delivering Plasmid DNA.” As described, the triblock copolymer p(ManEMA-b-DMAEMA-b-[DEAEMA-co-BMA]) was synthesized using controlled reversible addition-fragmentation chain transfer (RAFT) polymerization. See, e.g., Scheme 4. The final synthesized tribock copolymer contained a first polymannose-containing block for cell targeting, second polyDMAEMA block for functional association of plasmid DNA, and a third block containing DEAEMA and BMA to serve as the micelle-forming, pH-responsive, endosomolytic block. See Scheme 4. As described, the pH-responsiveness of the triblock copolymers was validated using dynamic light scattering (DLS) measurements and hemolysis assays. See FIGS. 9 and 10.

In a third embodiment, the polymer composition is a copolymer comprising repeating units having pendant carbohydrate groups and repeating units having pendant functional groups suitable for associating a therapeutic agent and/or a diagnostic agent to the copolymer. In this embodiment, the copolymer is a random copolymer having targeting and agent binding capacity. In one embodiment, the copolymer further includes repeating units having neutral pendant groups.

In a specific embodiment, the invention provides a copolymer having formula (III):

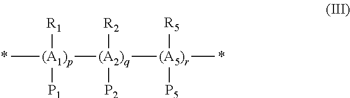

wherein

A₁(R₁)(P₁) is a first repeating unit, wherein A₁ is a backbone of the repeating unit, R₁ is a substituent of the repeating unit selected from the group consisting of hydrogen and C1-C8 alkyl optionally substituted with one or more fluorine atoms, and P₁ is a pendant group comprising a carbohydrate,

A₂(R₂)(P₂) is a second repeating unit, wherein A₂ is a backbone of the repeating unit, R₂ is a substituent of the repeating unit selected from the group consisting of hydrogen and C1-C8 alkyl optionally substituted with one or more fluorine atoms, and P₂ is a pendant group comprising a functional group suitable for associating a therapeutic or diagnostic agent to the block copolymer, and

A₅(R₅)(P₅) is a third repeating unit, wherein A₅ is a backbone of the repeating unit, R₅ is a substituent of the repeating unit selected from the group consisting of hydrogen and C1-C8 alkyl optionally substituted with one or more fluorine atoms, and P₅ is a neutral pendant group.

In some embodiments of the copolymer having formula (III), p is the mole fraction of the repeating unit, wherein p is from 0.01 to 0.99. In some embodiments, p is from 0.01 to 0.5. In some embodiments, p is from 0.1 to 0.4. In some embodiments, p is from 0.2 to 0.3. In some embodiments, p is from 0.2 to 0.8. In some embodiments, p is from 0.3 to 0.7. In some embodiments, p is from 0.4 to 0.6. In some embodiments, p is from 0.5 to 0.99. In some embodiments, p is from 0.6 to 0.9. In some embodiments, p is from 0.7 to 0.8.

In some embodiments of the copolymer having formula (III), q is the mole fraction of the repeating unit, wherein q is from 0.01 to 0.99. In some embodiments, q is from 0.01 to 0.5. In some embodiments, q is from 0.1 to 0.4. In some embodiments, q is from 0.2 to 0.3. In some embodiments, q is from 0.2 to 0.8. In some embodiments, q is from 0.3 to 0.7. In some embodiments, q is from 0.4 to 0.6. In some embodiments, q from 0.5 to 0.99. In some embodiments, q is from 0.6 to 0.9. In some embodiments, q is from 0.7 to 0.8.

In some embodiments of the copolymer having formula (III), r is the mole fraction of the repeating unit in the second block, wherein r is from 0.0 to 0.99. In some embodiments, r is from 0.0 to 0.5. In some embodiments, r is from 0.1 to 0.4. In some embodiments, r is from 0.2 to 0.3. In some embodiments, r is from 0.2 to 0.8. In some embodiments, r is from 0.3 to 0.7. In some embodiments, r is from 0.4 to 0.6. In some embodiments, r from 0.5 to 0.99. In some embodiments, r is from 0.6 to 0.9. In some embodiments, r is from 0.7 to 0.8.

It will be appreciated that the present invention includes embodiments of any combination of the ranges and subranges described herein for each of the variables of formula (III).

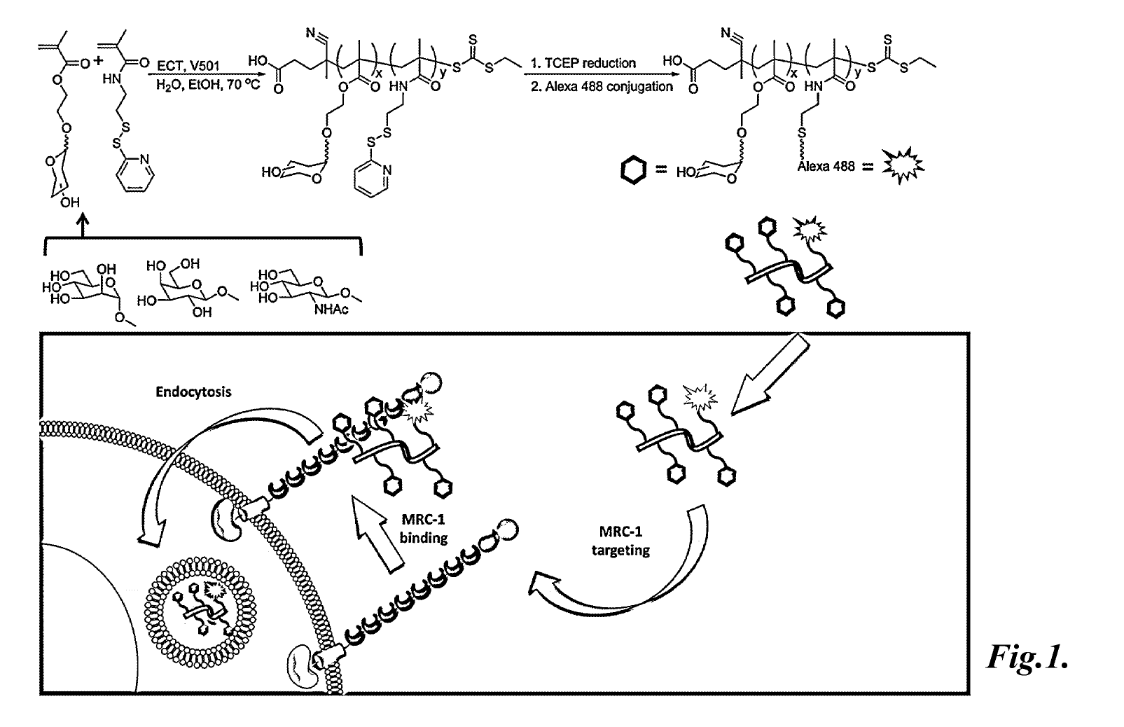

In an illustrative example, described below in the representative embodiment entitled, “Synthesis and Characterization of Carbohydrate Containing Copolymers Exhibiting Lectin Specific, Receptor Mediated Uptake,” copolymers were synthesized that comprised pendant mannose-, N-acetylglucosamine-, or galactose groups. For each carbohydrate-containing copolymer, the carbohydrate groups were in random combination with a pyridyl disulfide comonomer, specifically pyridyl disulfide ethyl methacrylamide (PDSEMA). See, e.g., FIG. 1. The PDSEMA was incorporated during the polymerization at a 10% molar feed ratio to provide a randomly-placed pendant functional group to serve as a conjugatable handle for attachment of a maleimide-containing fluorophore. As described, the resulting copolymers exhibited the dual functions of carbohydrate-based cell targeting/uptake and retaining a detectable label, namely a fluorophore to facilitate the monitoring of copolymer uptake.

In a fourth embodiment, the polymer composition is a block copolymer comprising a first block comprising repeating units having pendant carboxylic acid groups and a second block comprising repeating units having pendant carbohydrate groups and repeating units having pendant functional groups suitable for associating a therapeutic or diagnostic agent to the block copolymer. In one embodiment, the first block further comprises repeating units having neutral pendant groups. In another embodiment, the second block further comprises repeating units having neutral pendant groups. In a further embodiment, the first and second blocks further comprise repeating units having neutral pendant groups.

In a specific embodiment, the invention provides a copolymer having formula (IV):

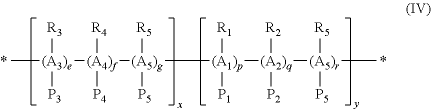

wherein

A₁(R₁)(P₁) is a second block repeating unit, wherein A₁ is a backbone of the repeating unit, R₁ is a substituent of the repeating unit selected from the group consisting of hydrogen and C1-C8 alkyl optionally substituted with one or more fluorine atoms, and

P₁ is a pendant group comprising a carbohydrate, A₂(R₂)(P₂) is a second block repeating unit, A₂ is a backbone of the repeating unit, R₂ is a substituent of the repeating unit selected from the group consisting of hydrogen and C1-C8 alkyl optionally substituted with one or more fluorine atoms, and P₂ is a pendant group comprising a functional group suitable for associating a therapeutic agent to the block copolymer,

A₃(R₃)(P₃) is a first block repeating unit, wherein A₃ is a backbone of the repeating unit, R₃ is a substituent of the repeating unit selected from the group consisting of hydrogen and C1-C8 alkyl optionally substituted with one or more fluorine atoms, and P₃ is a pendant group comprising a carboxylic acid,

A₄(R₄)(P₄) is a first block repeating unit, wherein A₄ is a backbone of the repeating unit, R₄ is a substituent of the repeating unit selected from the group consisting of hydrogen and C1-C8 alkyl optionally substituted with one or more fluorine atoms, and P₄ is a pendant group comprising an amine, and

A₅(R₅)(P₅) is a repeating unit, wherein A₅ is a backbone of the repeating unit, R₅ is a substituent of the repeating unit selected from the group consisting of hydrogen and C1-C8 alkyl optionally substituted with one or more fluorine atoms, and P₅ is a pendant group comprising a neutral group.

In some embodiments of the copolymer having formula (IV), e is the mole fraction of the repeating unit in the first block, wherein e is from 0.0 to 1.0. In some embodiments, e is from 0.01 to 0.5. In some embodiments, e is from 0.1 to 0.4. In some embodiments, e is from 0.2 to 0.3. In some embodiments, e is from 0.2 to 0.8. In some embodiments, e is from 0.3 to 0.7. In some embodiments, e is from 0.4 to 0.6. In some embodiments, e is from 0.5 to 1.0. In some embodiments, e is from 0.6 to 0.9. In some embodiments, e is from 0.7 to 0.8.

In some embodiments of the copolymer having formula (IV), f is the mole fraction of the repeating unit in the first block, wherein f is from 0.0 to 1.0, wherein e+f is at least 0.01. In some embodiments, f is from 0.0 to 0.5. In some embodiments, f is from 0.1 to 0.4. In some embodiments, f is from 0.2 to 0.3. In some embodiments, f is from 0.2 to 0.8. In some embodiments, f is from 0.3 to 0.7. In some embodiments, f is from 0.4 to 0.6. In some embodiments, f is from 0.5 to 0.99. In some embodiments, f is from 0.6 to 0.9. In some embodiments, f is from 0.7 to 0.8.

In some embodiments of the copolymer having formula (IV), g is the mole fraction of the repeating unit in the first block, wherein g is from 0.0 to 0.99. In some embodiments, g is from 0.0 to 0.5. In some embodiments, g is from 0.1 to 0.4. In some embodiments, g is from 0.2 to 0.3. In some embodiments, g is from 0.2 to 0.8. In some embodiments, g is from 0.3 to 0.7. In some embodiments, g is from 0.4 to 0.6. In some embodiments, g is from 0.5 to 0.99. In some embodiments, g is from 0.6 to 0.9. In some embodiments, g is from 0.7 to 0.8.

In some embodiments of the copolymer having formula (IV), p is the mole fraction of the repeating unit in the second block, wherein p is from 0.01 to 0.99. In some embodiments, p is from 0.01 to 0.5. In some embodiments, p is from 0.1 to 0.4. In some embodiments, p is from 0.2 to 0.3. In some embodiments, p is from 0.2 to 0.8. In some embodiments, p is from 0.3 to 0.7. In some embodiments, p is from 0.4 to 0.6. In some embodiments, p from 0.5 to 0.99. In some embodiments, p is from 0.6 to 0.9. In some embodiments, p is from 0.7 to 0.8.

In some embodiments of the copolymer having formula (IV), q is the mole fraction of the repeating unit in the second block, wherein q is from 0.01 to 0.99. In some embodiments, q is from 0.01 to 0.5. In some embodiments, q is from 0.1 to 0.4. In some embodiments, q is from 0.2 to 0.3. In some embodiments, q is from 0.2 to 0.8. In some embodiments, q is from 0.3 to 0.7. In some embodiments, q is from 0.4 to 0.6. In some embodiments, q from 0.5 to 0.99. In some embodiments, q is from 0.6 to 0.9. In some embodiments, q is from 0.7 to 0.8.

In some embodiments of the copolymer having formula (IV), r is the mole fraction of the repeating unit in the second block, wherein r is from 0.0 to 0.99. In some embodiments, r is from 0.0 to 0.5. In some embodiments, r is from 0.1 to 0.4. In some embodiments, r is from 0.2 to 0.3. In some embodiments, r is from 0.2 to 0.8. In some embodiments, r is from 0.3 to 0.7. In some embodiments, r is from 0.4 to 0.6. In some embodiments, r is from 0.5 to 0.99. In some embodiments, r is from 0.6 to 0.9. In some embodiments, r is from 0.7 to 0.8.

In some embodiments of the copolymer having formula (IV), x is the mole fraction of the first block, wherein x is from 0.01 to about 0.99. In some embodiments, x is from 0.01 to 0.5. In some embodiments, x is from 0.1 to 0.4. In some embodiments, x is from 0.2 to 0.3. In some embodiments, x is from 0.2 to 0.8. In some embodiments, x is from 0.3 to 0.7. In some embodiments, x is from 0.4 to 0.6. In some embodiments, x from 0.5 to 0.99. In some embodiments, x is from 0.6 to 0.9. In some embodiments, x is from 0.7 to 0.8. In some embodiments, x is about 0.5. In some embodiments, x is 0.5.

In some embodiments of the copolymer having formula (IV), y is the mole fraction of the second block, wherein y is from 0.01 to about 0.99. In some embodiments, y is from 0.01 to 0.5. In some embodiments, y is from 0.1 to 0.4. In some embodiments, y is from 0.2 to 0.3. In some embodiments, y is from 0.2 to 0.8. In some embodiments, y is from 0.3 to 0.7. In some embodiments, y is from 0.4 to 0.6. In some embodiments, y is from 0.5 to 0.99. In some embodiments, y is from 0.6 to 0.9. In some embodiments, y is from 0.7 to 0.8. In some embodiments, y is about 0.5. In some embodiments, y is 0.5.

It will be appreciated that the present invention includes embodiments of any combination of the ranges and subranges described herein for each of the variables of formula (IV).

The polymer compositions of the present invention are effective for targeted intracellular delivery of one or more therapeutic and/or diagnostic agents. In these embodiments, the polymer compositions of the invention further include an associated therapeutic agent and/or diagnostic agent. In some embodiments, the therapeutic agent and/or diagnostic agent is associated with at least one pendant group of the polymer composition.

Thus, the polymer compositions of the invention include the repeating units having pendant functional groups suitable for associating a therapeutic and/or diagnostic agent to the polymer. As used herein, the term “suitable for associating” refers to the pendant functional groups of the copolymers of the present invention that are capable of retaining any agent or moiety of interest in close association as a payload for intracellular delivery. The association between the one or more pendant functional group and the one or more associated agent can be established by any interaction including, by way of non-limiting example, one or more covalent bonds, one or more non-covalent interactions (e.g., ionic bonds, static forces, van der Waals interactions, combinations thereof, or the like), or a combination thereof. Any suitable conjugation method can be used, for example a large variety of conjugation chemistries are available (see, for example, Bioconjugation, Aslam and Dent, Eds, Macmillan, 1998 and chapters therein). In certain embodiments, the functional group is a thiol. In other embodiments, the functional group is a disulfide. In these embodiments, the agents (e.g., proteins and peptides) are reversibly covalently coupled (e.g., via disulfide linkages) to the polymer. In certain other embodiments, the functional groups are amine or cationic groups. In these embodiments, the agents (e.g., oligonucleotides) are reversibly ionically or electrostatically associated to the polymer.

In certain embodiments, one or more of a plurality the block copolymers is associated with a therapeutic agent. In some embodiments, one or more of a plurality of block copolymers is associated with a first therapeutic agent, and wherein one or more of the plurality of block copolymers is associated with a second therapeutic agent. In further embodiments, the block copolymers that are associated with the first therapeutic agent and the block copolymers that are associated with the second therapeutic agent are allowed to self assemble, resulting in a micellic assembly comprising a first and second associated therapeutic agent. Thus, in certain embodiments, one or more of the plurality of block copolymers is attached to a first therapeutic agent, and wherein one or more of the additional polymers is attached to a second therapeutic agent.

As used herein, “therapeutic agent” refers to an agent that, when administered to a subject, organ, tissue, or cell has a therapeutic effect and/or elicits a desired biological and/or pharmacological effect. In some embodiments, the therapeutic agent is an immunotherapeutic agent, wherein the agent elicits, facilitates, enhances, or otherwise influences the activation or performance of one or more immune cells. Suitable therapeutic agents include proteins, peptides, and oligonucleotides.

In specific embodiments, the therapeutic agent is a polynucleotide, an oligonucleotide, a gene expression modulator, a knockdown agent, an siRNA, an RNAi agent, a dicer substrate, an miRNA, an shRNA, an antisense oligonucleotide, an aptamer, or an aiRNA. Such nucleic acid-based therapeutic agents can serve various roles, such as to modulate endogenous gene expression in the target cell (e.g., knockdown expression via RNAi), encode signaling molecules, and encode antigens for internal processing and presentation via the target cell's endogenous major histocompatibility complex (MHC).

In some embodiments, the polynucleotide is an expression vector, e.g., mammalian expression vector. The expression vector typically comprises a complimentary DNA sequence (a “cDNA” or mini-gene) that is functionally linked to a promoter region such that the promoter drives expression of the cDNA. In certain instances, mammalian expression vectors also comprise a polyadenylation signal at the 3′ end of the cDNA. A promoter region is a nucleotide segment that is recognized by a RNA polymerase molecule, in order to initiate RNA synthesis (i.e., transcription), and may also include other transcriptional regulatory elements such as enhancers. Any number of transcriptional regulatory sequences may be used to mediate expression of linked genes in mammalian expression vectors. Promoters include but are not limited to retroviral promoters, other viral promoters such as those derived from HSV or CMV, and promoters from endogenous cellular genes. Mammalian expression vectors also typically have an origin of replication from E. coli to enable propagation as plasmids in bacteria.

As described in the representative embodiments below, in some embodiments the block copolymers are useful for forming micellic assemblies complexed with polynucleotides, such as plasmid DNA, for delivery into a cell or to an individual in need thereof. The nucleotide can be attached to a block copolymer provided herein in any manner suitable, e.g., by non-covalent association. For example, in certain embodiments, the micellic assembly's polycationic blocks (e.g., blocks containing functional pendant groups) associate with the plasmid DNA and complexes the DNA with the micellic assembly. In certain instances, polycations bind to and complex with plasmid DNA. In some embodiments, a micellic assembly comprising a polynucleotide complex is charge neutralized (e.g., the copolymer block comprising pendant functional groups and the polynucleotide are substantially charge neutralized). Depending on the length of the polynucleotide, the length of the polycationic block is optionally adjusted to provide charge neutralization for the polynucleotide. In some instances, charge-neutralization is achieved by addition of cations and/or polycations into the formulation.

In other embodiments, a conjugate of one or more therapeutic agent (e.g., oligonucleotide) with a block copolymer, wherein the copolymer is a unimer or present in an assembled micellic assembly, is prepared according to a process comprising the following two steps: (1) activating a modifiable end group (for example, 5′ or 3′-hydroxyl or) of an oligonucleotide using any suitable activation reagents, such as but not limited to 1-ethyl-3,3-dimethylaminopropyl carbodiimide (EDAC), imidazole, N-hydrosuccinimide (NHS) and dicyclohexylcarbodiimide (DCC), HOBt (1-hydroxybenzotriazole), p-nitrophenylchloroformate, carbonyldiimidazole (CDI), and N,N′-disuccinimidyl carbonate (DSC); and (2) covalently linking a block copolymer to the end of the oligonucleotide. In some embodiments' the 5′- or 3′-end modifiable group of an oligonucleotide is substituted by other functional groups prior to conjugation with the block copolymer. For example, hydroxyl group (—OH) is optionally substituted with a linker carrying sulfhydryl group (—SH), carboxyl group (—COOH), or amine group (—NH₂).

In certain embodiments, the therapeutic agent is a proteinaceous agent, such as a peptide, a polypeptide, a peptide dominant-negative protein, enzyme, hormone, antibody, or antibody fragment. In some embodiments, the proteinaceous agent can serve as an antigen, such as a polypeptide fragment that can be presented by the target cell via the cells endogenous major histocompatibility complex (MHC). In other embodiments, the proteinaceous agent can provide a signal to modulate the behavior of the target cell or the other cells interacting therewith.

Exemplary antibodies include polyclonal, monoclonal and recombinant antibodies; multi-specific antibodies (e.g., bispecific antibodies); humanized antibodies; murine antibodies; chimeric antibodies, such as, mouse-human, mouse-primate, primate-human monoclonal antibodies; and anti-idiotype antibodies, and may be any intact molecule or fragment thereof. As used herein, the term “antibody fragment” refers to a portion derived from or related to a full-length antibody, generally including the antigen binding or variable region thereof. Illustrative examples of antibody fragments include Fab, Fab′, F(ab)₂, F(ab′)₂ and Fv fragments, scFv fragments, diabodies, linear antibodies, single-chain antibody molecules and multispecific antibodies formed from antibody fragments. Specifically, “single-chain Fv” or “scFv” antibody fragment comprises the V_{H} and V_{L} domains of an antibody, wherein these domains are present in a single polypeptide chain. Generally, the Fᵥ polypeptide further comprises a polypeptide linker between the V_{H} and V_{L} domains, which enables the scFv to form the desired structure for antigen binding. As used herein, a “chimeric antibody” is a recombinant protein that contains the variable domains and complementarity-determining regions derived from a non-human species (e.g., rodent) antibody, while the remainder of the antibody molecule is derived from a human antibody. As used herein, a “humanized antibody” is a chimeric antibody that comprises a minimal sequence that conforms to specific complementarity-determining regions derived from non-human immunoglobulin that is transplanted into a human antibody framework. Humanized antibodies are typically recombinant proteins in which only the antibody complementarity-determining regions are of non-human origin.

Conjugation of proteinaceous therapeutic agents (e.g., a polypeptide) to the copolymer assemblies provided herein is achieved according to a variety of conjugation processes by a chemical reaction involving one or more of the functional groups of the proteinaceous therapeutic agent (e.g., a polypeptide) with one or more of the functional groups present in the copolymer (in its unimer form or as part of a micellic assembly, etc.). Polypeptide functional groups that are usually involved include but are not limited to amino, hydroxy, thiol, or carboxyl groups. Such groups can be present as a terminal group or present on the amino acid side chains. In some embodiments, the proteinaceous therapeutic agents are engineered to contain non-natural amino acids comprising special functional groups for formation of site-specific conjugates, e.g., azido groups for conjugation via “click” chemistry.

In certain embodiments, a plurality of therapeutic agents is associated with the copolymers of the present invention.

As used herein, “diagnostic agent” refers to agents or moieties that facilitate the observation, detection or monitoring of a biological state. A biological state can be, for example, a disease state, including the presence of an infectious agent or cancer, or a particular activation or activity of a cell-type. In some embodiments, the diagnostic agent is a diagnostic imaging agent, many of which are commonly known in the art. For example, an agent useful in imaging the mammalian vascular system includes, but is not limited to, position emission tomography (PET) agents, computerized tomography (CT) agents, magnetic resonance imaging (MRI) agents, nuclear magnetic imaging agents (NMI), fluoroscopy agents and ultrasound contrast agents. Such diagnostic agents include radioisotopes of such elements as iodine (1), including ¹²³I, ¹²⁵I, ¹³¹I etc., barium (Ba), gadolinium (Gd), technetium (Tc), including ⁹⁹Tc, phosphorus (P), including ³¹P, iron (Fe), manganese (Mn), thallium (Tl), chromium (Cr), including ⁵¹Cr, carbon (C), including ¹⁴C, or the like, fluorescently labeled compounds, or their complexes, chelates, adducts and conjugates. In other embodiments, the diagnostic agent is a marker gene that encode proteins that are readily detectable when expressed in a cell (including, but not limited to, β-galactosidase, green fluorescent protein, luciferase, and the like) and labeled nucleic acid probes (e.g., radiolabeled or fluorescently labeled probes). In some embodiments, covalent conjugation of diagnostics agents to the copolymer pendant functional groups provided herein is achieved according to a variety of conjugation processes known in the art. In other embodiments, the diagnostic agent is non-covalently associated with the copolymer pendant functional group provided herein by complexing with a chelating residue (e.g., a carboxylic acid residue) incorporated into the block copolymers. In some embodiments, a radiolabeled monomer (e.g., a ¹⁴C-labeled monomer) is incorporated into the polymeric backbone of the copolymers. In some embodiments, a copolymer pendant functional group associated with a diagnostic agent comprises a targeting moiety.

The preferable mole ration of the pendant functional groups within the copolymer or copolymer block is dependent on various factors, such as the nature of the therapeutic/diagnostic agent to be attached, the nature of the pendant carbohydrate group, and the ultimate goal of use for the copolymer. In consideration of these and other factors, persons of ordinary skill in the art can optimize the mole ratio of the functional groups. For example, one would consider the nature of the species to be tethered, the amount of said species that needs to be tethered, and the nature of the chemistry to be employed in the coupling reaction. An example is the use of pyridyl disulfide functional groups present in the copolymer to attach biomacromolecules such as short interfering RNA (siRNA). For example, reactive groups can comprise from 0.01 to 0.2 mole fraction of the copolymer (or copolymer block). For example, in the context of the embodiment described above in formula (I), with a mole fraction of 0.2 between a pyridyl disulfide functional groups (in the second block) and the aggregate of the neutral and carbohydrate groups is indicative of 20% of the entire polymer being pyridyl disulfide functional groups. According to formula (I), this could be represented in the formula where c is 0.4 and both x and y are 0.5, so as to maintain 20% pyridyl disulfide functional groups. The functional group can be integrated into the copolymer 1) as a polymerizable moiety, 2) post polymerization through chemical modification of another functional group present in the polymer, or 3) through the use of prefunctionalized RAFT chain transfer agents (CTA)s. If a prefunctionalized chain transfer agent is employed, there will be a single functional group localized at the alpha and/or omega chain terminus depending on the chemical nature of the CTA. These functional groups can be the same or different. In one implementation of this technology, the functional group is located at the alpha chain terminus (i.e., the end of the chain where polymerization begins). This distinction is important because if the polymer self-assembles to form of heterogenous morphology in aqueous conditions (e.g., micelles or nanoparticles) the functional group may be sequestered from aqueous phase if the functional group is integrated on a phase separated region of the copolymer.

The polymer compositions of the invention target specific cells. The targeting capability is imparted to the polymer compositions by the polymers' pendant carbohydrate groups. Carbohydrates are known to play roles in cell to cell interactions of immune cells as well as marking foreign pathogens for immune cell recognition. Cell-surface lectins are capable of recognizing carbohydrates with high specificity imparted by their carbohydrate recognition domains (CRDs).

Because individual CRDs typically exhibit low binding affinity towards a monosaccharide, receptors such as C-type lectins typically cluster multiple binding domains in close proximity to increase overall affinity towards oligosaccharides, which is termed the “glycoside cluster effect”. To mimic the multivalent binding capabilities of natural saccharides, the copolymers of the present invention provide a plurality of well-defined carbohydrate pendant groups to facilitate multivalent interactions with the carbohydrate receptors on the cell surfaces. The simultaneous engagement of multiple CRDs on the receptor in turn promotes the uptake of the copolymer complex into the cell. Thus, the presentation of carbohydrates in the copolymers of the present invention offers a means to mediate therapeutic and/or diagnostic agent uptake through cell-surface carbohydrate receptors, while maintaining biocompatibility.

Various cell types have unique cell-surface carbohydrate receptor profiles, which presents an opportunity to specifically target those cells for uptake through their carbohydrate receptors. For example, dendritic cells (DCs) and macrophages express a range of C-type lectins, such as macrophage mannose receptor (MMR) and langerin, which are able to internalize bound material. MMR is an endocytic C-type lectin found on macrophages and subsets of DCs that contains eight individual CRDs. Ligands capable of multivalent binding through simultaneous engagement of multiple CRDs on MMR are potent facilitators of macrophage-specific uptake. While mannose displays the highest affinity toward MMR CRDs, other sugars such as fucos, N-acetyl glucosamine (GlcNAc), and glucose, also engage MMR CRDs. Langerin is expressed predominantly on a subpopulation of skin DCS, namely Langerhans cells (LCs) and displays a high affinity for mannose. Additionally, langerin also binds fucose and GlcNAc-containing carbohydrates. Antigens that are typically internalized via langerin on LCs are capable of mediating potent CD4+ and CD8+ T cell responses, which presents a potent target for vaccine design.

Accordingly, suitable carbohydrates for inclusion in the copolymers of the present invention include any carbohydrate capable of binding cell surface receptors. Such carbohydrate groups can include monosaccharides groups, disaccharide groups, oligosaccharide groups, and polysaccharide groups. Monosaccharides, whether appearing as a discrete pendant group or as part of a longer pendant group, include base groups with 2, 3, 4, 5, 6, or more, carbon atoms, including any isomer thereof. As non-limiting examples, representative pendant carbohydrate groups can consist of or comprise mannose, galactose, fucose, N-acetyl glucosamine, sialic acid, and combinations thereof. As used herein, the terms “glycan” and “glycopolymer” may be used to refer to the disclosed carbohydrates and carbohydrate-containing polymers, respectively.

In certain embodiments, the polymer compositions of the invention include carboxylic acid pendant groups that impart pH responsiveness to the composition. For these embodiments, representative repeating units having pendant carboxylic acid groups such as C1-C8 alkyl acrylic acid repeating units, acrylic acid repeating units, and combinations thereof. As used herein the term “C1-C8 alkyl acrylic acid repeating units” refers to repeating units derived from C1-C8 alkyl acrylic acids (e.g., methacrylic acid, ethyl acrylic acid, propyl acrylic acid).

The polymer compositions of the invention that include carboxylic acid pendant groups can further include repeating units having neutral pendant groups. The neutral pendant groups can be utilized to control the hydrophobicity of the polymer composition, thereby controlling polymer composition properties. For these embodiments, representative repeating units having neutral pendant groups include acrylic acid C1-C8 ester repeating units, C1-C8 alkyl acrylic acid C1-C8 ester repeating units, and combinations thereof. As used herein the term “acrylic acid C1-C8 ester repeating units” refers to repeating units derived from acrylic acid C1-C8 esters (e.g., methyl acrylate, ethyl acrylate, propyl acrylate, butyl acrylate). As used herein the term “C1-C8 alkyl acrylic acid C1-C8 ester repeating units” refers to repeating units derived from C1-C8 alkyl acrylic acid C1-C8 esters (e.g., methyl methacrylate, methyl ethacrylate, ethyl methacrylate, propyl methacrylate, butyl methacrylate).

The polymer compositions of the invention that include carboxylic acid pendant groups can further include repeating units having amine pendant groups. For these embodiments, representative repeating units having pendant amine groups are derived from monomer that include amine groups such as dimethylamino ethyl methacrylic acid (DMAEMA).

The polymer compositions of the invention that include carboxylic acid pendant groups can also include repeating units having neutral pendant groups and repeating units having amine pendant groups.

For polymer compositions that include repeating units having pendant carboxylic acid groups and repeating units having pendant amine groups, in certain embodiments, these compositions include a substantially equimolar amount of repeating units having pendant carboxylic acid groups and repeating units having pendant amine groups.

In another aspect, the invention provides a nanoparticle. The nanoparticle comprises a plurality of copolymers of the invention that include repeating units having membrane destabilizing functionality (e.g., pendant carboxylic acid groups). At least a portion of the copolymers making up the nanoparticle include an associated therapeutic and/or diagnostic agent. The nanoparticle is serum-stable nanoparticle. In certain embodiments, the nanoparticle is a micelle or micellic assembly. In these embodiments, the polymers assemble into micelles having a predominantly hydrophobic core and a hydrophilic shell.

In a further aspect, the invention provides pharmaceutical compositions. The pharmaceutical composition comprises a pharmaceutically acceptable carrier and at least one polymer compositions, copolymers or nanoparticle of the invention.

Formulations comprising the polymer compositions of the invention (i.e., copolymers or nanoparticles) can be pharmaceutical compositions. Such pharmaceutical compositions can comprise, for example, a composition of the invention and a pharmaceutically acceptable excipient.

In some embodiments, the polymer composition is administered to a patient in any suitable manner, e.g., with or without stabilizers, buffers, and the like, to form a pharmaceutical composition. In some embodiments, the polymer composition is formulated and used as tablets, capsules or elixirs for oral administration, suppositories for rectal administration, sterile solutions, suspensions or solutions for injectable administration, and any other suitable compositions.

In some embodiments, pharmaceutical compositions comprising the polymer composition are administered systemically. As used herein, “systemic administration” means in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. In some embodiments, the compositions are administered topically.

In some embodiments, the compositions are prepared for storage or administration and include a pharmaceutically effective amount of the pH-responsive polymer composition in a pharmaceutically acceptable carrier or diluent. Any acceptable carriers or diluents are optionally utilized herein. Specific carriers and diluents and are described, e.g., in Remington's Pharmaceutical Sciences, Mack Publishing Co., A. R. Gennaro Ed., 1985. As used herein, the term “pharmaceutically acceptable carrier” means a non-toxic, inert solid, semi-solid or liquid filler, diluent, encapsulating material or formulation auxiliary of any type. In some embodiments, the pharmaceutical compositions provided herein are administered to humans and/or to animals, orally, rectally, parenterally, intracistemally, intravaginally, intranasally, intraperitoneally, topically (as by powders, creams, ointments, or drops), bucally, or as an oral or nasal spray.

In various embodiments, liquid dosage forms for oral administration include pharmaceutically acceptable emulsions, microemulsions, solutions, suspensions, syrups, and elixirs. In addition to the active ingredients, the liquid dosage forms optionally further contain inert diluents or excipients.

The polymer compositions of the invention are used for intracellular delivery of a therapeutic agent (i.e., protein or peptide). The composition can be exposed to and contacted with a cell surface (e.g., via directed carbohydrate targeting). In certain embodiments, the composition is introduced into an endosomal membrane within the cell, for example, through endocytosis and, in some embodiments, through receptor mediated endocytosis. The endosomal membrane is destabilized (e.g., by a membrane destabilizing copolymer), thereby delivering the therapeutic agent (e.g., protein or peptide) to the cytosol of the cell.

Accordingly, in another aspect, the invention provides a method for introducing one or more of a therapeutic and/or diagnostic agent into a cell, comprising contacting a cell with at least one copolymer as described herein.

In another aspect, a method of modifying the level of expression of an endogenous protein is provided. In some embodiments, the method comprises silencing or reducing the level of expression of the endogenous protein. In one embodiment, the method includes the step of contacting a cell with a copolymer having a therapeutic agent associated therewith, wherein the therapeutic agent modulates the expression of the endogenous protein in the cell. For example, as described herein, the therapeutic agent can be a polynucleotide, such as an siRNA, a dicer substrate, an miRNA, an shRNA, an antisense oligonucleotide, an aptamer, or an aiRNA or the like, which function to reduce the endogenous mRNA levels encoding the protein.

The polymers provided herein can be synthesized using any methods known those of skill in the art. In certain embodiments, the polymers are synthesized using reversible addition-fragmentation chain transfer (RAFT) polymerization. The RAFT method is described further in the representative embodiments below, and it will be appreciated that polymers formed using the RAFT method will have minimized polymer polydispersity and compositional drift compared to similar polymers formed using other methods.

The following describes representative embodiments of the present invention. Specifically, the following describes the synthesis and characterization of copolymers containing carbohydrate and functional groups for lectin specific uptake in macrophages; the synthesis and characterization of triblock copolymers that self assemble into micellic assemblies that exhibit pH-responsive disruption and cell specific targeting; and the application of the triblock copolymer design to specifically target DCs and deliver thereto plasmid DNA polyepitope cancer antigens. These embodiments are intended to illustrate various aspects of the invention and are not intended to imply any limitations.

### Synthesis and Characterization of Carbohydrate-Containing Copolymers Exhibiting Lectin-Specific, Receptor-Mediated Uptake

### 1. Rationale and Overview

Targeting cell populations via endogenous carbohydrate receptors is an appealing approach for drug delivery. However, to be effective, this strategy requires the production of high affinity carbohydrate ligands capable of engaging with specific cell-surface lectins. To develop materials that exhibit high affinity towards these receptors, carbohydrate-containing copolymers were synthesized that displayed pendant carbohydrate moieties from carbohydrate-functionalized monomer precursors via reversible addition-fragmentation chain transfer (RAFT) polymerization. These copolymers were fluorescently labeled via pendant functional groups and used to determine macrophage-specific targeting both in vitro and in vivo. Mannose- and N-acetylglucosamine-containing copolymers were shown to specifically target mouse bone marrow-derived macrophages (BMDMs) in vitro in a dose-dependent manner as compared to a galactose-containing copolymer (30- and 19-fold higher uptake, respectively). In addition, upon macrophage differentiation, the mannose-containing copolymer exhibited enhanced uptake in M2-polarized macrophages, an anti-inflammatory macrophage phenotype prevalent in injured tissue. This carbohydrate-specific uptake was retained in vivo, as alveolar macrophages demonstrated 6-fold higher internalization of mannose-containing copolymer, as compared to a galactose-containing copolymer, following intratracheal administration in mice. These results demonstrate the successful synthesis of a class of functional RAFT copolymers capable of macrophage-type specific targeting and uptake, both in vitro and in vivo. The results provide significant promise for successful integration of the carbohydrate-containing copolymers into future targeted drug delivery systems.

### 2. Introduction

The targeted delivery of small molecule drugs and biologics continues to be a major objective towards improving therapeutic efficiency through the mitigation of off-target effects and reduction in required dose. However, few delivery carriers are capable of recognizing target cell-specific ligands while avoiding nonspecific cellular uptake. To overcome this limitation, carbohydrate-based materials have been investigated due to their biocompatibility, target specificity, and ability to facilitate receptor-mediated uptake through cell-surface lectins (carbohydrate-binding proteins).

Macrophages are an attractive therapeutic target because they play an important role in the inflammatory response and wound healing. A number of macrophage subsets have been described that are associated with distinct phenotypes. Classically activated macrophages (M1) are critical for host defense and arise from microbial byproducts or Th1 cytokines. In response to acute injury, the predominant macrophage phenotype is pro-inflammatory (M1). In contrast, the alternatively activated macrophages (M2) are stimulated by Th2 cytokines and are important in injury resolution and wound healing, including anti-inflammatory activity. However, an overexuberant M1 macrophage response can result in collateral tissue damage and impaired wound healing. Likewise, while M2 macrophages are important for appropriate wound healing, excess M2 response can result in tissue fibrosis. Indeed, dysregulated macrophage function is associated with a wide range of conditions including chronic ulcers, allergic asthma, atherosclerosis, autoimmune disorders, and fibrotic diseases. The ability to target and modulate macrophage function has important therapeutic implications. Carbohydrates are known to play a significant role in the inflammatory response through mediating cell-cell recognition of immune cells, including macrophages. Macrophages are a promising target for carbohydrate-based therapeutics as they express carbohydrate binding receptors that internalize bound material via receptor-mediated endocytosis. One such carbohydrate binding receptor is the macrophage mannose receptor (MMR), an endocytic protein that is highly expressed on macrophages, including the alveolar macrophage. The mannose receptor mediates the uptake and internalization of extracellular ligands including potentially harmful extracellular glycoproteins with terminal mannose, fucose, or N-acetylglucosamine, and pathogens with high densities of mannose on their surface. The murine mannose receptor, MRC-1 (CD206), contains eight extracellular C-type lectin-like domains (CTLDs). Simple monosaccharides exhibit low affinities towards single MRC-1 CTLDs, with dissociation constants in the low millimolar range. Many natural glycans enhance these weak binding events by clustering multiple glycosides together, thereby allowing multivalent interactions to be made with a multidomain lectin receptor (e.g. the mannose receptor) leading to a significant increase in overall avidity. Ligands capable of exhibiting this multivalent behavior through simultaneous engagement of multiple CTLDs on the mannose receptor are potent facilitators of macrophage-specific uptake. While mannose displays the highest affinity toward mannose receptor CTLDs, other sugars (e.g. fucose, N-acetylglucosamine and glucose) are also recognized by these lectin-like domains.

Through the presentation of multiple pendant carbohydrates, the synthetic copolymers of the present invention, described herein, provide a promising platform to facilitate mannose receptor-mediated binding and subsequent endocytosis. While similar compounds have been previously synthesized to probe lectin-carbohydrate binding behavior, the compounds employed were not structurally well-defined. Therefore, as described herein, well-defined, homogenous carbohydrate-containing copolymers capable of multivalent interactions were successfully prepared from vinyl-functionalized carbohydrates (glycomonomers). These glycomonomers can be polymerized through free radical polymerization to yield a copolymer with pendant carbohydrates. Use of reversible addition-fragmentation chain transfer (RAFT) polymerization for this copolymer synthesis offers precise control over the reaction, resulting in predictable molecular weights, narrow molecular weight distributions, and the ability to develop complex polymer architectures. The successful RAFT polymerization of a glycomonomer was first demonstrated by using glucose-functionalized methacrylate monomer in aqueous conditions; the resultant material exhibited a low polydispersity and displayed “living” properties characteristic of the RAFT technique. Additionally, through the modification of the chain transfer agent (CTA) and the incorporation of comonomers, facile telechelic and pendant polymer functionalization is achievable. By combining the versatility of the RAFT process with carbohydrate synthetic techniques, structurally complex glycosylated materials capable of mimicking the multivalent binding activity of biological carbohydrate compounds can be realized, including carbohydrate-containing copolymer micelles, stars, nanoparticles, “clickable” constructs, and glycosylated block copolymers. By displaying multiple functional carbohydrates, these materials can be used to target specific cell populations via carbohydrate-dependent uptake mechanisms, which allows for the design of diagnostic and therapeutic glycosylated constructs.

Glycosylation of drug delivery vehicles has been explored as a means to access alveolar macrophages in vivo, a cell implicated in the pathogenesis of pulmonary conditions. For example, it has been demonstrated that mannosylating liposomes enhanced their uptake by rat alveolar macrophages in vivo following intratracheal administration. However, there has yet to be a study systemically evaluating the uptake of well-defined, multivalent carbohydrate materials by macrophages both in vitro and in pulmonary tissue. As demonstrated herein, a family of fluorescently-labeled carbohydrate-containing copolymers capable of macrophage-specific targeting was developed employing synthesized glycomonomers and RAFT polymerization. The demonstrated copolymer uptake is dose-, time-, cell-type-, and carbohydrate-dependent in vitro and the carbohydrate-specific uptake is retained in vivo.

### 3. Materials and Methods

### 3.1 Materials

Materials were purchased from Sigma-Aldrich unless otherwise specified. 4,4′-Azobis(4-cyanovaleric acid) (V501) was obtained from Wako Chemicals USA, Inc. 4-Cyano-4-(ethylsulfanylthiocarbonyl)sulfanylvpentanoic acid (ECT) and Pyridyl disulfide ethyl methacrylamide (PDSEMA) was synthesized as described previously.

3.2 General Synthesis of Glycomonomers

All commercially available chemicals were used without additional purification unless otherwise noted. ¹H and ¹³C NMR were obtained at 500 MHz and 125 MHz on a Bruker AV-500 NMR. Mass spectra (MS) were recorded on Bruker APEX Qe 47e Fourier transform mass spectrometer. Chemical shifts are reported in parts per million downfield relative to tetramethylsilane (TMS, 0.00 ppm) and coupling constant are reported in Hertz (Hz). The following abbreviations are used for the multiplicities: s=singlet; d=doublet; t=triplet; q=quartet; m=multiplet; and br=broad.

### 3.2.1 Synthesis of 2-O-(α-D-mannosyl)hydroxyethyl methacrylate (1-3, 1-4)

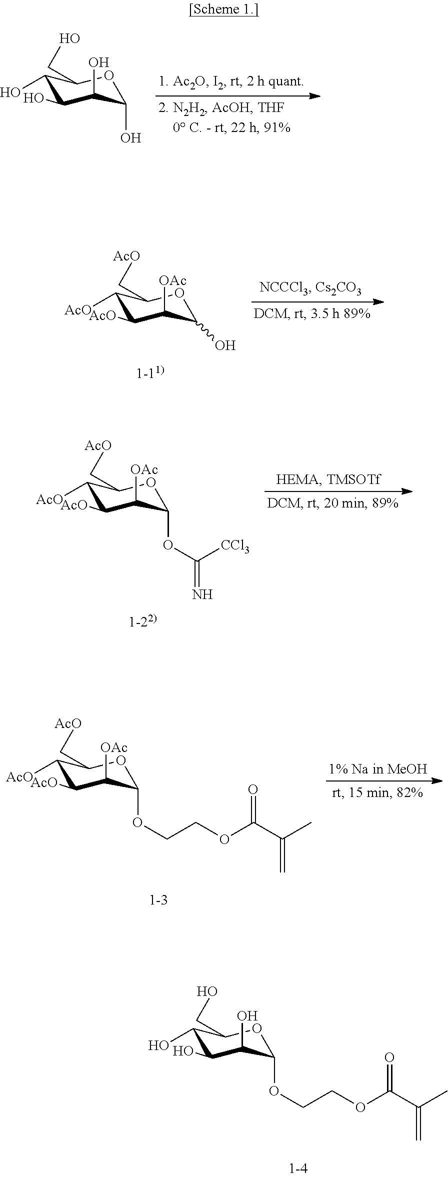

### Synthesis of 2-O-((2′,3′,4′,6′-tetra-O-acetyl)-α-D-mannosyl)hydroxyethyl methacrylate (1-3)

TMSOTf (10 μL) was added to a mixture of (2′,3′,4′,6′-tetra-O-acetyl)-α-D-mannosyl trichloroacetimidate (1 g, 2.0 mmol) and 2-hydrocyethyl methacrylate (0.31 mL, 2.4 mmol) in dichloromethane (3 mL) at room temperature. The reaction mixture was stirred at room temperature for 20 min and then quenched by the addition of triethylamine. The product (0.83 g, 89% yield) was obtained after removal of solvent under reduced pressure followed by purification through silica column chromatography.

¹H NMR (500 MHz, CDCl₃) δ 6.16 (s, 1H), 5.64 (dd, J=3.1, 1.5 Hz, 1H), 5.40 (dd, J=10, 3.4 Hz, 1H), 5.33 (d, J=10 Hz, 1H), 5.30 (dd, J=3.6, 1.8 Hz, 1H), 4.90 (d, J=1.4 Hz, 1H), 4.38-4.36 (m, 2H), 4.32 (dd, J=12.2, 5.4 Hz, 1H), 4.13 (dd, J=12.2, 2.2 Hz, 1H), 4.07-4.03 (m, 1H), 3.97-3.93 (m, 1H), 3.82-3.79 (m, 1H), 2.19 (s, 3H), 2.13 (s, 3H), 2.07 (s, 3H), 2.02 (s, 3H), 1.99 (s, 3H); ¹³C NMR (125 MHz, CDCl₃) δ 170.44, 169.82, 169.71, 169.56, 166.91, 135.86, 125.89, 97.35, 69.26, 68.86, 68.48, 65.96, 65.73, 63.06, 62.28, 20.68, 20.52, 20.50 (2 C), 18.12; HRMS-ESI (m/z): [M+Na]⁺ calcd. for C₂₀H₂₈NaO₁₂, 483.1478. found, 483.1466.

### Synthesis of 2-O-(α-D-mannosyl)hydroxyethyl methacrylate (1-4)

To a solution of 2-O-((2′,3′,4′,6′-tetra-O-acetyl)-α-D-mannosyl)hydroxyethyl methacrylate (1.2 g, 2.6 mmol) in MeOH (5 mL) was added 1% Na in MeOH solution (0.1 mL) and the mixture was stirred at 25° C. for 15 min. The reaction mixture was neutralized with AcOH. The desired product (0.62 g, 82% yield) was obtained after evaporation of solvent under reduced pressure, followed by purification through silica column chromatography (10% MeOH in dichloromethane).

¹H NMR (500 MHz, CD₃OD) δ 6.14 (s, 1H), 5.66 (s, 1H), 4.83 (s, 1H), 4.39-4.29 (m, 2H), 4.00-3.96 (m, 1H), 3.85 (dd, J=11.8, 1.9 Hz, 1H), 3.83 (br, 1H), 3.77-3.72 (m, 2H), 3.71 (dd, J=9.2, 3.2 Hz, 1H), 3.64 (dd, J=9.5, 9.2 Hz, 1H), 3.60-3.56 (m, 1H), 1.96 (s, 3H); ¹³C NMR (125 MHz, CD₃OD) δ 165.91, 134.72, 123.76, 98.72, 71.83, 69.68, 69.19, 65.55, 63.49, 62.06, 59.96, 15.70; HRMS-ESI (m/z): [M+Na]⁺ calcd. for C₁₂H₂₀NaO₈, 315.1056. found, 315.1046.

### 3.2.2 Synthesis of 2-O-(β-D-galactosyl)hydroxyethyl methacrylate (2-3, 2-4)

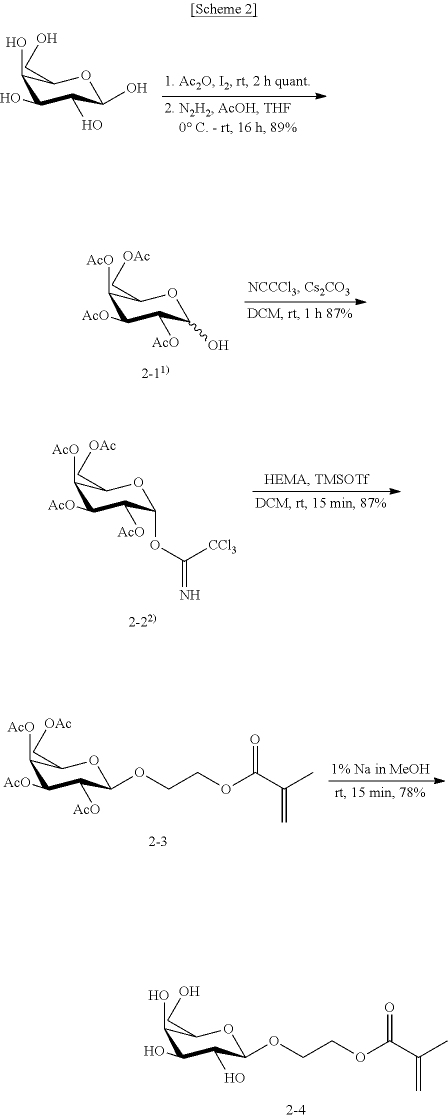

### Synthesis of 2-O-((2′,3′,4′,6′-tetra-O-acetyl)-β-D-galactosyl)hydroxyethyl methacrylate (2-3)

To a mixture of (2′,3′,4′,6′-tetra-O-acetyl)-β-D-galactosyl trichloroacetimidate (1.5 g, 3.1 mmol) and 2-hydrocyethyl methacrylate (0.46 mL, 3.7 mmol) in dichloromethane (5 mL) was added TMSOTf (10 μL) at room temperature. The reaction mixture was stirred at room temperature for 15 min and then quenched by the addition of triethylamine. The product (1.22 g, 87% yield) was obtained after removal of solvent under reduced pressure followed by purification through silica column chromatography.

¹H NMR (500 MHz, CDCl₃) δ 6.15 (s, 1H), 5.62 (dd, J=1.5, 1.5 Hz, 1H), 5.42 (d, J=2.6 Hz, 1H), 5.26 (dd, J=10.4, 8.0 Hz, 1H), 5.04 (dd, J=10.4, 3.4 Hz, 1H), 4.57 (d, J=8 Hz, 1H), 4.36-4.30 (m, 2H), 4.21-4.13 (m, 2H), 4.10-4.06 (m, 1H), 3.94 (dd, J=6.7, 6.7, 1H), 3.88-3.84 (m, 1H), 2.17 (s, 3H), 2.07 (s, 3H), 2.04 (s, 3H), 2.00 (s, 3H), 1.98 (s, 3H); ¹³C NMR (125 MHz, CDCl₃) δ 170.07, 170.04, 169.80, 169.04, 166.79, 135.95, 125.60, 100.95, 70.68, 70.52, 68.53, 67.17, 66.96, 63.31, 61.17, 20.38 (3 C), 20.30, 18.03; HRMS-ESI (m/z): [M+Na]⁺ calcd. for C₂₀H₂₈NaO₁₂, 483.1478. found, 483.1464.

### Synthesis of 2-O-(β-D-galactosyl)hydroxyethyl methacrylate (2-4)

To a solution of Synthesis of 2-O-((2′,3′,4′,6′-tetra-O-acetyl)-(3-D-galactosyl)hydroxyethyl methacrylate (1 g, 2.2 mmol) in MeOH (5 mL) was added 1% Na in MeOH solution (0.1 mL) and the mixture was stirred at 25° C. for 15 min. The reaction mixture was neutralized with AcOH. The desired product (0.49 g, 78% yield) was obtained after evaporation of solvent under reduced pressure followed by purification through silica column chromatography (10% MeOH in dichloromethane).

¹H NMR (500 MHz, CD₃OD) δ 6.15 (s, 1H), 5.65 (dd, J=1.5, 1.5, 1H), 4.38-4.35 (m, 2H), 4.31 (d, J=7.6, 1H), 4.15-4.11 (m, 1H), 3.90-3.85 (m, 2H), 3.78-3.72 (m, 2H), 3.57-3.52 (m, 2H), 3.49 (dd, J=9.7, 3.2 Hz, 1H), 1.96 (d, J=0.9, 3H); ¹³C NMR (125 MHz, CD₃OD) δ 167.51, 136.25, 125.17, 103.87, 75.31, 73.51, 71.04, 68.86, 67.20, 63.96, 61.06, 17.11; HRMS-ESI (m/z): [M+Na]⁺ calcd. for C₁₂H₂₀NaO₈, 315.1056. found, 315.1049.

### 3.2.3 Synthesis of 2-O-(N-acetyl-β-D-glucosaminosyl)hydroxyethyl methacrylate (3-3, 3-4)

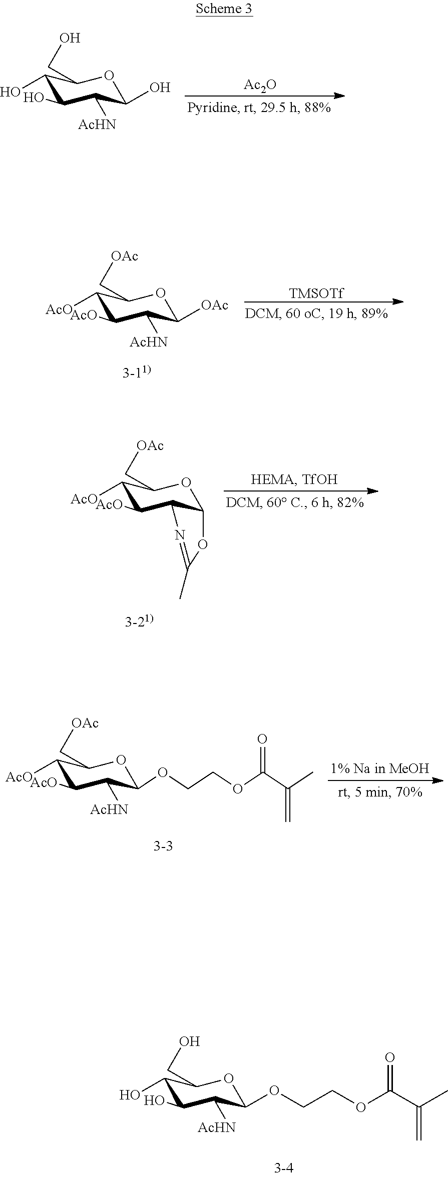

### Synthesis of 2-O-((3′,4′,6′-tri-O-acetyl)-N-acetyl-β-D-glucosaminosyl)hydroxyethyl methacrylate (3-3)

To a mixture of 2-methyl-2-(3,4,6-tri-O-acetyl-1,2-dideoxy-D-glucopyrano)-[2,1]-2-oxazoline (1.2 g, 3.6 mmol) and 2-hydrocyethyl methacrylate (0.68 mL, 5.4 mmol) in dichloromethane (10 mL) was added TfOH (28 μL, 0.32 mmol) at room temperature. The reaction mixture was stirred at 60° C. for 6 h and then quenched by the addition of triethylamine. The product (1.46 g, 87% yield) was obtained after removal of solvent under reduced pressure followed by purification through silica column chromatography.

¹H NMR (500 MHz, CDCl₃) δ 6.15 (s, 1H), 5.62 (d, J=1.3, 1H), 5.52 (d, J=8.6, 1H), 5.31 (dd, J=9.5, 9.5, 1H), 5.10 (dd, J=9.5, 9.5, 1H), 4.79 (d, J=8.3, 1H), 4.46-4.42 (m, 1H), 4.30 (dd, J=12.3, 4.7, 1H), 4/26-4.22 (m, 1H), 4.17 (dd, J=12.5, 1.9, 1H), 4.08-4.05 (m, 1H), 3.92-3.86 (m, 2H), 3.74-3.71 (m, 1H), 2.11 (s, 3H), 2.06 (s, 6H), 1.97 (s, 3H), 1.94 (s, 3H); ¹³C NMR (125 MHz, CDCl₃) δ 170.63, 170.53, 170.35, 169.29, 167.03, 135.93, 125.74, 100.11, 72.33, 71.50, 68.91, 66.98, 63.04, 62.20, 54.21, 22.91, 20.54, 20.50, 20.44, 18.07; HRMS-ESI (m/z): [M+Na]⁺ calcd. for C₂₀H₂₉NNaO₁₁, 482.1638. found, 482.1630.

### Synthesis of 2-O-(N-acetyl-β-D-glucosaminosyl)hydroxyethyl methacrylate (3-4)

To a solution of 2-O-((3′,4′,6′-tri-O-acetyl)-N-acetyl-β-D-glucosaminosyl)hydroxyethyl methacrylate (1 g, 2.2 mmol) in MeOH (3 mL) was added 1% Na in MeOH solution (0.1 mL) and the mixture was stirred at 25° C. for 5 min. The reaction mixture was neutralized with AcOH. The desired product (0.51 g, 70% yield) was obtained after evaporation of solvent under reduced pressure followed by purification through silica column chromatography (10% MeOH in dichloromethane).

¹H NMR (500 MHz, CD₃OD) δ 6.14 (d, J=0.6, 1H), 5.66 (dd, J=1.5, 1.5 Hz, 1H), 4.50 (dd, J=8.4, 1 Hz, 1H), 4.34-4.28 (m, 2H), 4.10-4.06 (m, 1H), 3.91 (d, J=5.4 Hz, 1H), 3.83-3.79 (m, 1H), 3.72-3.64 (m, 2H), 3.48 (dd, J=8.9, 0.5 Hz, 1H), 3.31-3.30 (m, 1H), 1.97 (d, J=1.4 Hz, 3H), 1.96 (d, J=0.7 Hz, 3H); ¹³C NMR (125 MHz, CD₃OD) δ 172.31, 167.35, 136.27, 125.18, 101.11, 76.62, 74.57, 70.69, 66.79, 63.59, 61.41, 55.90, 21.78, 17.16; HRMS-ESI (m/z): [M+Na]⁺ calcd. for C₁₄H₂₃NNaO₈, 356.1321. found, 356.1314.

### 3.3 Synthesis of Copolymers with Pendant Carbohydrates

The RAFT polymerization of the copolymers of the present invention was conducted in a heterogeneous solvent system of dH₂O/ethanol (3:1 vol:vol) at 70° C. with 15 wt % monomer under a nitrogen atmosphere for 4 h using ECT and V501 as the chain transfer agent (CTA) and radical initiator, respectively. The initial CTA to monomer molar ratio ([CTA]₀:[M]₀) was 50:1, the initial CTA to initiator molar ratio ([CTA]₀:[I]₀) was 20:1, and the initial molar feed ratios of glycomonomer to PDSEMA was 9:1. The resultant copolymer was isolated by dilution into dH₂O followed by lyophilization. The copolymer was further purified by redissolution into dH₂O, chromatographic separation with a PD-10 desalting column (GE Healthcare, Piscataway, N.J.), and further lyophilization to obtain the final polymer.

### 3.4 Carbohydrate-Containing Copolymer Characterization

Monomer conversion and incorporation were determined by ¹H-NMR (500 MHz, D₂O). Conversion was determined to be greater than 99% due to the absence of resonances associated with vinyl protons on the glycomonomer (δ 6.07) and PDSEMA (δ 5.77) following polymerization. Copolymer composition was calculated from an aromatic PDSEMA proton (δ 8.40) and a methylene proton vicinal to the ester group (δ 4.15 Man) or methine proton on the pyranose ring (δ 4.36 Gal and δ 4.51 GlcNAc). Molecular weights (Mₙ) and polydispersity indices (PDI) were determined by gel permeation chromatography (GPC) using a Viscotek GPCmax VE2001 and refractometer VE3580 (Viscotek, Houston, Tex.) with Tosoh TSK-GEL α-3000 (2×) and α-4000 columns connected in series (Tosoh Bioscience, Montgomeryville, Pa.). HPLC-grade N,N-dimethylacetamide (DMAc; 0.03% w/v LiBr; 0.05% w/v BHT) was used as the eluent at a flow rate of 0.85 mL/min while column temperature was maintained at 50° C. Absolute number average molecular weights were calculated from do/dc values that were determined for each carbohydrate-containing copolymer (pManEMA: 0.101, pGalEMA: 0.100, pGlcNAcEMA: 0.130). Incorporation of the pendant PDSEMA functional group was further validated by reduction of the carbohydrate-containing copolymers in the presence of Bond-Breaker TCEP solution (˜150 molar excess per polymer at 50 mM; Thermo Scientific, Rockford, Ill.) followed by spectroscopic measurement of liberated pyridine-2-thione (ε₃₄₃=8080 M cm⁻¹).

### 3.5 Fluorophore Labeling of Copolymers

Copolymers (˜10 mg mL⁻¹, in 0.1M sodium phosphate pH 7.4 with 0.15 M NaCl buffer) were incubated in immobilized TCEP disulfide reducing gel (˜10 molar excess per polymer; Thermo Scientific, Rockford, Ill.) for 2 h followed by elution with PBS. Alexa Flour 488 (AF488) C5 maleimide (10 mg mL⁻¹ in DMSO) was added to the reduced polymer in solution resulting in an approximately equimolar amount of fluorophore to reduced PDS groups and an overall polymer concentration of ˜2 mg mL⁻¹. The reaction proceeded overnight at room temperature followed by removal of excess fluorophore by a PD-10 desalting column and lyophilization. Fluorophore labeling efficiency was determined by spectroscopic measurement of the trithiocarbonate species on the copolymer end group (ε₃₁₀=20,000 M cm⁻¹) and the Alexa Fluor 488 (ε₄₉₅=73,000 M cm⁻¹).

### 3.6 Lectin Agglutination Assay

The ability of the carbohydrate-containing copolymers to bind a mannose-specific lectin, Concanavalin A (ConA), was assessed by an agglutination assay. 1 μM ConA was mixed with 10 μM copolymer (based on number of carbohydrate repeats) and the solution turbidity was measured by UV-Vis spectroscopy at 350 nm at one minute intervals for 30 minutes.

### 3.7 In vitro copolymer uptake assay

Female C57BL6 mice, from 8 to 12 weeks old, were maintained under specific pathogen-free conditions. Animal protocol was approved by University of Washington Institutional Animal Care and Use Committee.

Primary mouse lung fibroblasts (MLF) were isolated and cultured as previously described. The murine lung epithelial cell line MLE12 was obtained from American Type Culture Collection (ATCC). MLF and MLE12 uptake assays were performed in colorless DMEM supplemented with Glutamax (Life Technologies, Carlsbad, Calif.) and Nutridoma-SP (Roche, Base1, CH). Bone marrow derived macrophages (BMDM) were isolated from femurs and tibias of 8-12 week-old C57BL6 mice as previously described and cultured in RPMI-1640 containing 10% Fetal Bovine Serum and 30% L929-conditioned medium for 7-10 days.

To obtain differentiated macrophages, BMDMs (M0) were treated with 50 ng mL⁻¹ E. coli 0111:B4 LPS (Sigma, St. Louis, Mo.) for 24 hours (M1) or 20 ng mL⁻¹ each of IL-4 and IL-13 (Life Technologies, Carlsbad, Calif.) for 48 hours (M2). Cells were seeded in 12-well plates and allowed to adhere overnight. Cells were rinsed with DPBS, and then incubated with indicated concentration of Alexa488-labeled copolymers in colorless RPMI supplemented with Glutamax and Nutridoma-SP. For competition experiments, unlabeled copolymer was added 15 min prior to addition of labeled copolymer. At the end of incubation, the cells were washed and lifted with cold DPBS aided by cell scrapers. Internalized copolymer was measured as fluorescence intensity of the cells using Guava EasyCyte Plus System (Millipore, Hayward, Calif.) and data analyzed using CellQuest 2.0 (BD Biosciences, Franklin Lakes, N.J.). All experiments were performed in triplicate and repeated at least 3 times.

### 3.8 In Vivo Carbohydrate-Containing Copolymer Uptake Assay

8-12 week-old C57BL6 mice underwent intratracheal instillation with 10 μM Alexa-488-labeled copolymers or control (unlabeled) polymer in 50 μL DPBS. After 15-30 min, the lungs were lavaged with 1 mL of DPBS containing 0.6 mM EDTA. Bronchoalveolar lavage cells were centrifuged and rinsed to remove unincorporated polymers and resuspended in PBS for flow analysis by Guava as above. In some cases, BAL cells were spun onto microslides, and nuclei were counterstained with DAPI. Images were obtained using a Nikon Eclipse 80i microscope with a DS Camera Head DS-5M for fluorescent microscopy.

### 3.9 Statistical Analysis

Means of more than two groups of data were compared using one-way analysis of variance (ANOVA) for analysis of one independent variable or two-way ANOVA, for analysis of two independent variables, followed by Tukey's honestly significant difference (HSD) post hoc test. Student T-test was used for comparison of paired parametric data. For non-parametric data, Mann-Whitney's U test was performed. All tests were two-tailed and p values≦0.05 were considered significant.

### 4. Results and Discussion

### 4.1 Synthesis and Characterization of Carbohydrate-Containing Copolymers

Carbohydrates are attractive ligands for imparting biological targeting functionality to polymeric systems. Carbohydrate-ligands can be easily synthesized in large scale, are stable to indefinite storage at ambient temperatures, and can leverage low-affinity binding interactions through multivalency. Well-defined copolymers with pendant carbohydrate groups were prepared via the RAFT polymerization of synthesized glycomonomers, as generally illustrated in FIG. 1. Mannose (Man) and N-acetylglucosamine (GlcNAc) were selected as carbohydrate groups because of their known mannose receptor binding. Galactose (Gal) was also selected as a carbohydrate group due to its known lack of mannose receptor interactions. Each monosaccharide was functionalized with ethyl methacrylate (EMA), yielding the glycomonomers, referred to herein as ManEMA, GlcNAcEMA, and GalEMA, confirmed by NMR (not shown). These glycomonomers were polymerized via the RAFT technique. A pyridyl disulfide comonomer, pyridyl disulfide ethyl methacrylamide (PDSEMA), was incorporated into the polymerization (at a 10% molar feed ratio) to provide a pendant functional group, which provided a conjugatable handle for attachment of a maleimide-containing fluorophore. The resulting carbohydrate-containing copolymers were successfully synthesized under controlled conditions with consistent size and composition as determined by gel permeation chromatography (GPC) and ¹H-NMR spectroscopy (see Table 1). The carbohydrate copolymers are subsequently referred to as pManEMA, pGlcNAcEMA, and pGalEMA, depending on the pendant carbohydrate, and are understood to also contain the PDSEMA functional group. The RAFT-synthesized copolymers exhibited narrow molecular weight distributions with polydispersity indices (PDI) of 1.2 and resultant block lengths of 11400-13400 g mol⁻¹. The monomer incorporations of pyridyl disulfide groups per polymer were also similar between the copolymers (3.3-5.2). These findings demonstrate the first direct RAFT polymerization of the ManEMA and GlcNAcEMA glycomonomers used here.

TABLE 1
Molecular weights, compositions, conversions, and labeling efficiency of
carbohydrate-containing copolymers.

| | Mₙ^{a} | | % | PDS/ | Alexa488/ |
|---|---|---|---|---|---|
| Copolymer | (g/mol) | PDI^{a} | Conversion^{b} | polymer^{c} | polymer^{c} |
| pManEMA | 11400 | 1.2 | >99 | 5.2 | 0.93 |
| pGalEMA | 12200 | 1.2 | >99 | 4.5 | 0.95 |
| pGlcNAcEMA | 13100 | 1.2 | >99 | 3.3 | 1.2 |

^{a}Absolute number average molecular weights and polydispersity index (PDI) as determined by gel permeation chromatography (GPC).
^{b}Determined by 1H-NMR
^{c}Determined by UV-Vis spectroscopy; ratio represents number of pyridyl disulfide (PDS) groups per polymer chain

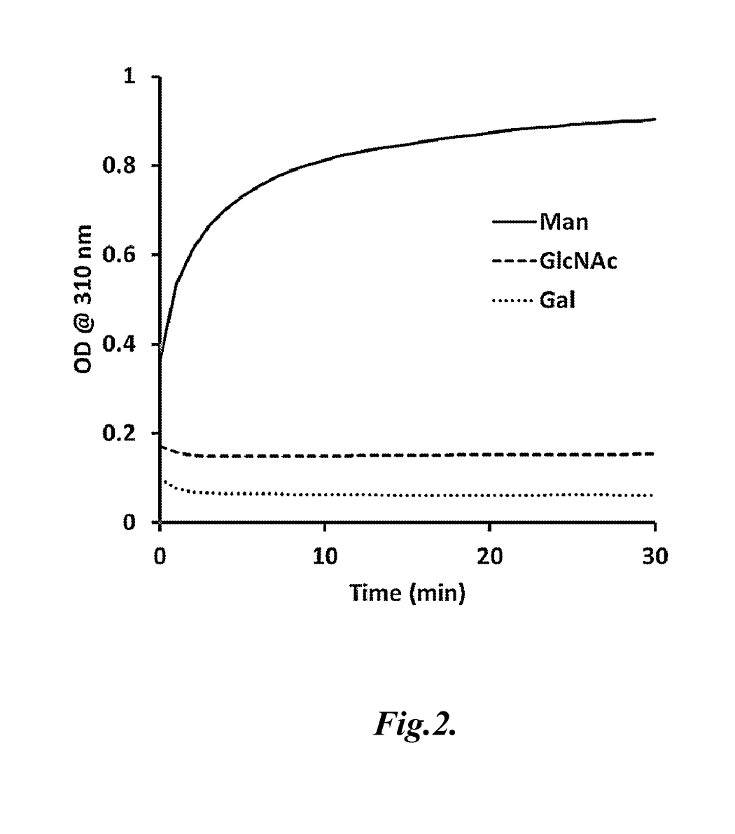

A maleimide-functionalized fluorophore (Alexa Fluor 488, AF488) was conjugated to the copolymers through the functional pyridyl disulfide (PDS) groups following reduction with tris(2-carboxyethyl)phosphine (TCEP). Labeling efficiency was similar among the three copolymers as determined by UV-Vis spectroscopy: 0.93-1.2 fluorophores/polymer (not shown). To initially determine lectin-binding activity, each carbohydrate-containing copolymer was incubated with Concanavalin A (ConA), a mannose-specific-binding lectin. pManEMA was found to agglutinate ConA, as measured by an increase in solution turbidity, showing that the material is capable of multivalent CRD-binding, a prerequisite for mannose receptor engagement (FIG. 2). The pGlcNAcEMA and pGalEMA copolymers did not induce ConA agglutination, demonstrating that the copolymer binding activity is carbohydrate-specific.

### 4.2 In vitro macrophage uptake of carbohydrate-containing copolymers

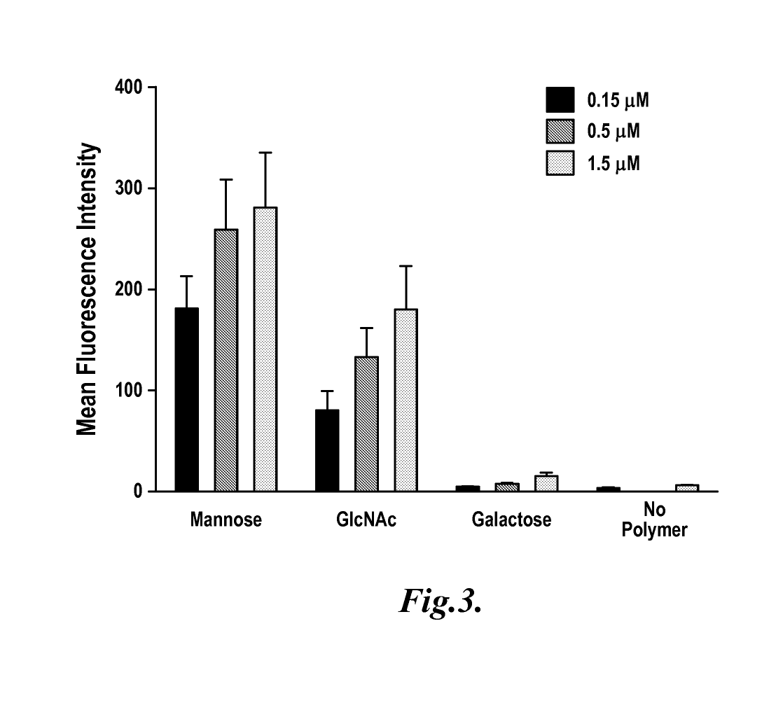

First, it was examined whether the carbohydrate-containing copolymers were differentially internalized by murine bone marrow-derived macrophages (BMDMs), a cell type known to express the mouse mannose receptor, MRC-1. BMDMs were incubated with increasing doses of AF488-labeled copolymers for varying lengths of time and cell uptake was assessed by flow cytometry and fluorescent microscopy. It was observed that the pManEMA and pGlcNAcEMA copolymers, but not the pGalEMA copolymer, were internalized efficiently by BMDMs in a time dependent fashion. The uptake of the pManEMA and pGlcNAcEMA copolymers were 30- and 19-fold higher than the pGalEMA copolymer, respectively. Significant uptake occurred by 15 min and increased up to 6 hours (FIG. 3 and data not shown). Mannose-binding protein, a soluble multi-domain C-type lectin in the same family as MRC-1, is known to exhibit carbohydrate-binding specificities defined by interactions with the vicinal, equatorial hydroxyl groups, C-3 and C-4, that are shared between several sugars. Therefore, the ability of polymerized Man and GlcNAc pendant groups to target macrophages is not surprising. BMDMs demonstrated a cytoplasmic punctate distribution of internalized pManEMA and pGlcNAcEMA copolymers, consistent with localization in the endosome/lysosome vesicles. No cell fluorescence above background was observed with the pGalEMA copolymer. A dose-dependent response on uptake was also observed for both pManEMA and pGlcNAcEMA with saturation of pManEMA uptake occurring at 0.5 μM (FIG. 3); no significant dose-dependent effects were observed for pGalEMA uptake.

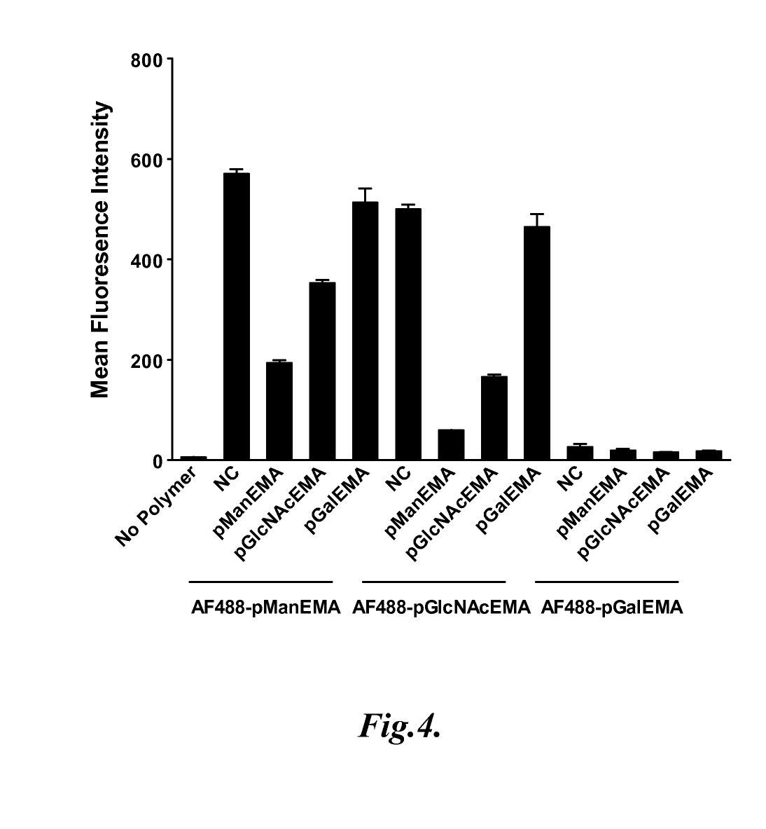

The effect of copolymer competition on uptake was assessed by incubating BMDMs with labeled copolymer in the presence of excess unlabeled copolymer. Competition with unlabeled pManEMA was more efficient than pGlcNAcEMA at attenuating uptake of labeled pManEMA and pGlcNAcEMA, suggesting that the binding affinity of MRC-1 towards pManEMA is higher than pGlcNAcEMA (FIG. 4). This finding is consistent with the mannose receptor's stronger affinity for Man pendant groups over GlcNAc pendant groups. The minimal uptake of pGalEMA by BMDMs was likely due to nonspecific macropinocytosis, supported by the lack of competition from any of the copolymers.

### 4.3 In Vitro Uptake of Carbohydrate-Containing Copolymers by Polarized Macrophages

Next, the ability of the carbohydrate-containing copolymers to exhibit specificity towards a polarization state of macrophages was assessed. Macrophages can be polarized into a classic “pro-inflammatory” (M1) or alternative “pro-resolution” (M2) state, depending on the local environment of cytokines and other immune-stimulating compounds. Macrophages can be differentiated in vitro by LPS to the M1 (“classically” activated) state, which results in the secretion of pro-inflammatory cytokines. Incubation with IL-4 and IL-13 leads to differentiation into M2 (“alternatively” activated) macrophages, which are considered a pro-resolution and anti-inflammatory phenotype. M2 macrophages secrete pro-fibrotic cytokines, such as tumor growth factor β (TGFβ) and platelet-derived growth factor (PDGF), that act on nearby fibroblasts to promote a fibroproliferative response. M2 macrophages also secrete matrix metalloproteases (MMPs) and tissue inhibitor of metalloproteinases (TIMPs) that regulate matrix remodeling. Additionally, they produce chemokines that attract other inflammatory cells (e.g. monocytes and dendritic cells) that subsequently clear apoptotic cells and debris, dampening the inflammatory response. Due to the orthogonal roles played by the M1 and M2 macrophage phenotype, differential targeting of activated macrophages is attractive for therapeutic drug delivery applications. Human alveolar macrophages adopting a M2 polarization are believed to play an important role in the pathogenesis of pulmonary fibrosis, highlighting this phenotype as a potential clinical target.

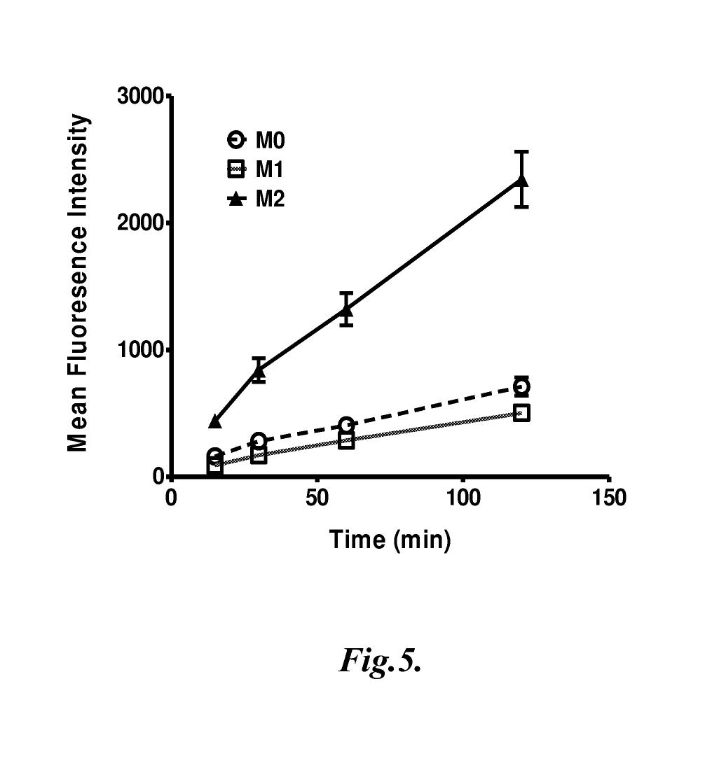

BMDMs were cultured in either LPS or IL-4/IL-13 to obtain either the M 1 or M2 polarization, respectively. Macrophages activated by the alternative pathway (M2) showed increased internalization of the pManEMA copolymer, whereas classically activated macrophages (M1) had similar internalization of the pManEMA copolymer as naïve macrophages (M0) (FIG. 5). These differences were retained over a two-hour time course. At this later time point, M2 macrophages had internalized pManEMA at an approximately 3- and 5-fold higher level than M0 and M1 macrophages, respectively.

M2 macrophages have higher expression of mannose receptor, providing further support that this receptor is facilitating the uptake of carbohydrate-containing copolymers. As previously described, the ability to distinguish macrophage subsets is of important clinical utility for fibrotic disease states.

### 4.4 In Vitro Cell-Specific Uptake of Carbohydrate-Containing Copolymer

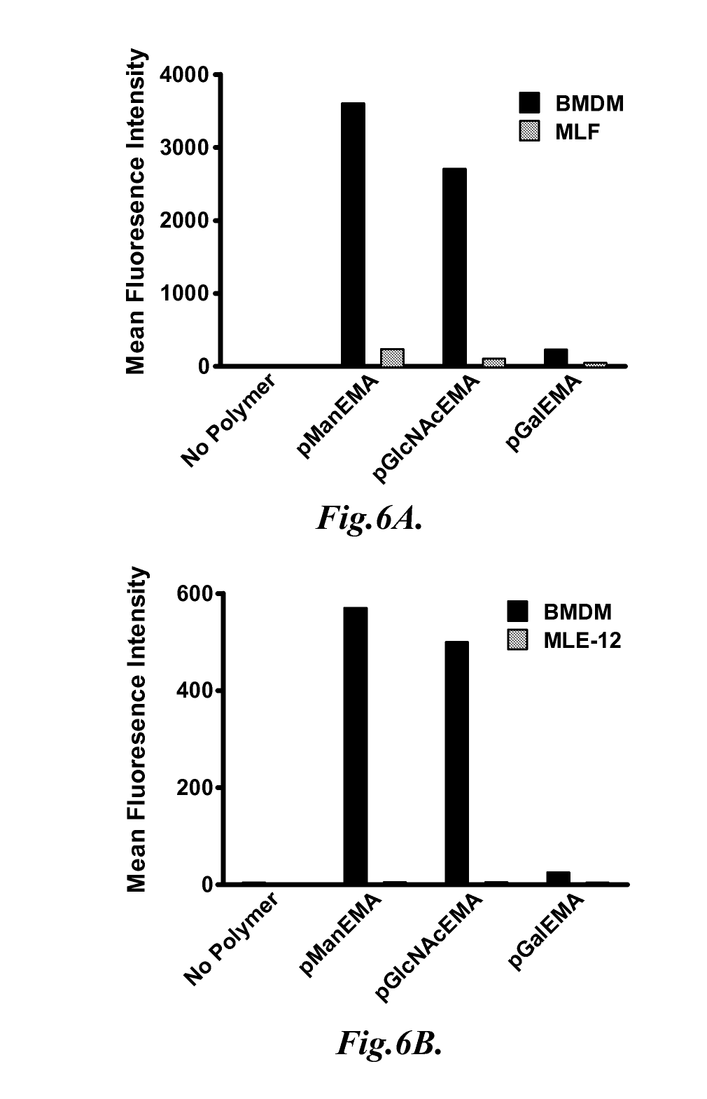

To determine cell-type specificity, uptake and internalization of labeled carbohydrate-containing copolymers by BMDMs, primary mouse lung fibroblasts (MLF) and the murine lung epithelial cell line, MLE-12, were compared. The latter two cell-types were selected as they are representative of the cell phenotypes encountered in the lung, the target site of the in vivo study (described below in section 4.5). Neither MLF nor MLE-12 had significant internalization of any of the copolymers at any timepoint examined (FIG. 6), whereas BMDMs had significant uptake of the pManEMA and pGlcNAcEMA copolymers. These results are consistent with mannose receptor-mediated uptake of the copolymers by macrophages.

### 4.5 In Vivo Macrophage Uptake of Carbohydrate-Containing Copolymers

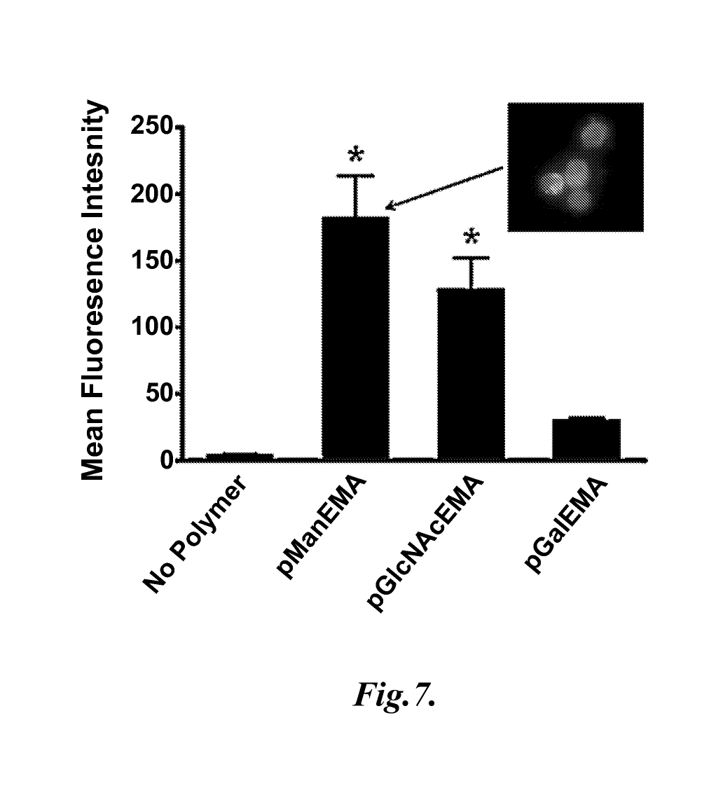

To determine whether macrophage internalization of the carbohydrate-containing copolymers retained the same carbohydrate specificity in vivo, Alexa 488-labeled copolymers were administered intratracheally in normal mice and measured uptake by alveolar macrophages. Bronchoalveolar lavage (BAL) was performed at different timepoints following administration, and BAL cells were analyzed for uptake of the copolymers. Greater than 90% of the BAL cells were alveolar macrophages, as verified by staining with a macrophage cell surface marker (F4/80, data not shown). Alveolar macrophages had similar in vivo uptake of copolymers as BMDM uptake in vitro. Specifically, the pManEMA and pGlcNAcEMA copolymers were readily internalized by alveolar macrophages as early as 30 minutes after instillation whereas the pGalEMA copolymer had minimal internalization (FIG. 7). Flow cytometric analysis showed that uptake of pManEMA was up to 6-folds higher than pGalEMA at 15 min (data not shown). Ex vivo examination of alveolar macrophage demonstrated a similar punctate distribution pattern as observed in BMDMs (FIG. 7, inset). These findings are consistent with previous work examining the uptake of mannosylated liposomes by alveolar macrophages following intratracheal administration in rats, namely, demonstrations that adding mannose to liposomes resulted in a 2.2 fold increase in uptake over a 24 hour period as compared to bare liposomes, and that a significant increase occurs in the internalization of mannosylated liposomes versus unmodified liposomes after as little as two hours. The results presented here demonstrate that these functional carbohydrates can remain active within the complex biological milieu found within the lung as pendant groups in a copolymer.

### 5. Conclusion

Synthetic carbohydrate-containing copolymers provide a promising platform to leverage mannose receptor-mediated endocytosis for the intracellular delivery of therapeutic cargo into specific macrophage populations in the lung. In this study, structurally well-defined copolymers were synthesized via RAFT polymerization and shown to specifically engage macrophages both in vitro and in vivo in a carbohydrate-dependent manner. It is demonstrated for the first time that macrophages activated by the alternative pathway (M2) showed increased internalization of mannose-containing copolymers and, to a lesser degree, N-acetylglucosamine-containing copolymers, whereas classically activated macrophages (M1) exhibit similar internalization of copolymers as naïve macrophages (M0). This finding is consistent with the reported up-regulation of mannose receptor expression in macrophages in response to IL-4 and IL-13 stimulation.

Developing a drug delivery platform capable of directly targeting polarized alveolar macrophages represents a promising strategy to treat pulmonary inflammatory conditions. Demonstration of specific uptake of the pManEMA copolymer by alveolar macrophages in vivo is promising for future therapeutic applications. Local delivery via intratracheal instillation has the advantage of less systemic toxicity and off-target effects, lower doses, and better drug stability. Moreover, local delivery of therapeutics to the lung is a feasible option in intubated patients suffering from acute respiratory distress syndrome. For example, the pyridyl disulfide functional group provides a handle on these copolymers that can be conjugated to other maleimide- or thiol-functionalized compounds, e.g. small molecule drugs and biological macromolecules. Coupled with the versatility of RAFT-based polymer synthesis, the carbohydrate-containing copolymers are a promising strategy for the design of targeted polymeric drug delivery systems.

### Evaluation and Characterization of Carbohydrate-Containing Copolymer-Mediated Targeting for Copolymer Micelles Delivering Plasmid DNA

### 1. Rationale and Overview

As demonstrated above, the RAFT-synthesized carbohydrate-containing copolymers provide a promising platform to leverage mannose receptor-mediated endocytosis for the intracellular delivery of therapeutic and diagnostic cargo in immune-related cells. Therefore, the role mannosylation of the biologic compound carrier would facilitate multivalent engagement of the C-type lectin, MMR, on dendritic cells (DCs) leading to receptor-mediated endocytosis. Targeting DCs is of therapeutic interest as they are potent mediators of immune responses, providing a powerful ally in vaccination strategies. The approach described herein modifies a prior synthetic scheme used to generate diblock copolymer micelles, which incorporate a micelle-forming, pH-responsive endosomolytic block with a functional block for DNA delivery. This diblock polymer is modified to further include with a carbohydrate-containing copolymer component (e.g., a block), resulting in triblock copolymers. To adapt the synthesis to the required organic conditions, acetylated glycomonomers were utilized necessitating a post-polymerization saponification reaction. A mannosylated triblock copolymer was prepared and thoroughly characterized. The ability of the triblock copolymer to condense pDNA, retain pH-responsive characteristics, and binding mannose-specific lectins were validated. Furthermore, preliminary studies demonstrated that the triblock copolymers exhibited specificity for murine bone marrow-derived dendritic cells (BMDCs). Assays to optimize the transfection and reduce cytotoxicity of the triblock copolymers in multiple target DC types are also described.

### 2. Introduction

The safe, efficacious delivery of DNA into a cell requires the use of carriers to facilitate cellular entry and nuclear localization while protecting the nucleic acid from enzymatic degradation. While polycation-based delivery systems are capable of exhibiting high transfection activities in vitro, their primary mode of cellular internalization, i.e., nonspecific adsorptive pinocytosis, limits their utility as in vivo nucleic acid carriers. Imparting carbohydrate-mediated targeting, termed “glycotargeting”, to these systems overcomes this shortcoming. As described above, cell-surface lectins are capable of recognizing carbohydrates with high specificity and can internalize adsorbed material through receptor-mediated endocytosis following multivalent engagement of carbohydrate recognition domains (CRDs). By directly targeting these lectins through the glycosylation of nucleic acid carriers, the uptake of the genetic cargo can be enhanced in specific cell populations.

Functionalizing polymeric gene delivery systems with a carbohydrate, e.g., mannose, presents an opportunity to selectively target delivery of genetic payloads for immune-modulation purposes because of the ability of the carbohydrate to recognize a range of C-type lectins, notably the macrophage mannose receptor (MMR), on the surfaces of macrophage and dendritic cell (DC) subsets. DCs are pivotal mediators of adaptive immune responses as they act as antigen presentation cells (APCs), priming T cells to elicit antigen-specific effector functions. Thus, delivering antigenic material to these APCs represents a promising strategy to initiate potent immune responses in a variety of vaccine applications.

Because an individual CRD exhibits low binding affinity towards a monosaccharide, C-type lectins typically cluster multiple binding domains in close proximity to increase overall affinity towards oligosaccharides, termed the “glycoside cluster effect”. As described above, to mimic the multivalent binding capabilities of natural saccharides, synthetic strategies to produce structurally well-defined carbohydrate-containing copolymers have been developed through the application of the reversible addition-fragmentation chain transfer (RAFT) process. These constructs contain pendant carbohydrate functionalities and can be incorporated into a variety of complex polymer architectures, including block copolymers.

In this embodiment, the development of triblock copolymers via RAFT is described, wherein the triblock copolymers comprise a targeting block with pendant carbohydrate groups, a delivery block with functional pendant groups, and micelle-forming, pH-responsive endosomolytic block. This approach is an extension of previous development of diblock copolymer micelles that exhibit a unique mode of endosomolytic activity, whereby they transition from micelles into membrane-interactive unimers at low pH. The membrane-interactive unimers that form the micelle-forming, pH-responsive endosomolytic block facilitate release from endosomes following cellular internalization. This activity was exploited to deliver condensed DNA (via the functional delivery block) to murine monocytic and dendritic cells in vitro. This design is presently modified through incorporation of a copolymer block containing pendant mannose groups, resulting in a triblock copolymer architecture. Triblock copolymers have previously been investigated as nonviral DNA vectors. Notable among these designs is the use of an ABC triblock copolymer consisting of targeting lactosylated poly(ethylene glycol), pH-responsive polyamine, and DNA-condensing polyamine segments. These DNA carriers demonstrated specific cell uptake in hepatocytes via the asialoglycoprotein receptor and higher transfection efficiencies as compared to related diblock copolymers. Here, the ability of glycotargeted triblock copolymers to complex with DNA and to transfect MR-expressing dendritic cells in an efficacious manner is evaluated.

### 3. Materials and Methods

3.1 Materials

Chemicals and all materials were supplied by Sigma-Aldrich (St Louis, Mo.) unless otherwise specified. 2,2′-Azobis(4-methoxy-2.4-dimethyl valeronitrile) (V70) and 1,1′-Azobis(cyclohexane-1-carbonitrile) (V40) were obtained from Wako Chemicals USA, Inc. (Richmond, Va.). Spectra/Por 7 standard regenerated cellulose dialysis tubing was obtained from Spectrum Labs (Rancho Dominguez, Calif.). pDNA gWizGFP was obtained from Aldevron LLC (Fargo, N. Dak.). Lipofectamine 2000 (LF) was obtained from Invitrogen (Carlsbad, Calif.). ECT was synthesized as previously described. Dimethylaminoethyl methacrylate (DMAEMA), diethylaminoethyl methacrylate (DEAEMA), and butyl methacrylate (BMA) were distilled prior to use. Acetylated mannose ethyl methylacrylate (AcManEMA) was synthesized according to methods described above. Diblock copolymers were prepared following procedures described previously. DC2.4 (murine dendritic cell line) cells were maintained in RPMI 1640 medium supplemented with 1% penicillin-streptomycin (GIBCO) and 10% fetal bovine serum (FBS, Invitrogen), Bone marrow-derived dendritic cells (BMDCs) were isolated according to previous procedures described below.

### 3.2 Synthesis of Poly(AcManEMA) Macro Chain Transfer Agent (pAcManEMA macroCTA)

The RAFT polymerization of the acetylated mannose macroCTA was conducted in dioxane at 30° C. with 40 wt % monomer under a nitrogen atmosphere for 18 h using ECT and V70 as the chain transfer agent (CTA) and radical initiator, respectively. The initial CTA to monomer molar ratio ([CTA]₀:[M]₀) was 25:1 and the initial CTA to initiator molar ratio ([CTA]₀:[I]₀) was 20:1. The resultant polymer was isolated by precipitation into cold hexanes. The polymer was then redissolved in acetone and subsequently precipitated into cold hexanes (×3) and dried overnight in vacuo.

### 3.3 Diblock Polymerization of DMAEMA from pAcManEMA macroCTA

A second polyDMAEMA block was polymerized using the isolated pAcManEMA macroCTA. The same protocol employed for the macroCTA was followed to prepare the p(AcManEMA-b-DMAEMA) diblock macroCTA with the exception of a ([CTA]₀:[M]₀) of 65:1.

### 3.4 Triblock Copolymerization of DEAEMA and BMA from p(AcManEMA-b-DMAEMA) macroCTA

DEAEMA and BMA were added to the macroCTA dissolved in dioxane at 40 wt %. The initial molar feed ratio of DEAEMA:BMA was 3:2 (40 mol % BMA). [M]₀/[ATA]₀ and [CTA]₀/[I]₀ were 100:1 and 20:1, respectively. Following the addition of V40 the solutions were purged with nitrogen for 30 min and allowed to react at 90° C. for 6 h. The resultant triblock copolymers were isolated by precipitation into cold hexanes. The precipitated polymers were then redissolved into acetone and precipitated into cold hexanes (×3) and dried overnight in vacuo.

### 3.5 Gel Permeation Chromatography (GPC)

Gel permeation chromatography (GPC) was used to determine molecular weights and polydispersities (M_{w}/Mₙ, PDI) of the macroCTA, diblock, and triblock copolymers. SEC Tosoh TSK-GEL R-3000 and R-4000 columns (Tosoh Bioscience, Montgomeryville, Pa.) were connected in series to a Agilent 1200 series (Agilent Technologies, Santa Clara, Calif.), refractometer Optilab-rEX and triple-angle static light scattering detector miniDAWN TREOS (Wyatt Technology, Dernbach, Germany). HPLC-grade DMF containing 0.1 wt. % LiBr at 60° C. was used as the mobile phase at a flow rate of 1 ml/min. The molecular weights of each polymer were determined using a multi-detector calibration based on do/dc values calculated separately for each homopolymer and copolymer composition.

### 3.6 Saponification of p(AcManEMA-b-DMAEMA-b-[DEAEMA-co-BMA])

To remove the acetyl groups and liberate the native sugar conformation, the dry triblock polymer was added to a solution of 1 wt % sodium methoxide in anhydrous methanol at an approximate copolymer concentration of 50 mg/mL. After 1 hour incubation at room temperature, the solution was neutralized with acetic acid to a pH of ˜7 and dialyzed against deionized water using 1000 MWCO tubing. The solution was lyophilized to obtain the final deprotected triblock copolymer: p(ManEMA-b-DMAEMA-b-[DEAEMA-co-BMA]).

### 3.7 Concanavalin A (ConA) Agglutination Assay

A stock solution of ConA was initially prepared in HEPES buffered saline (supplemented with MgCl₂ and CaCl₂). Triblock copolymer was added to a diluted ConA solution to obtain the following final concentrations: [ConA]=1 μM and [carbohydrate repeats in copolymer]=50 μM. At this point, the mixture was quickly vortexed and measured by UV-Vis spectroscopy. These measurements were repeated at one minute intervals for 30 min. For the competitively displaced (or “spiked”) sample, a solution of α-D-mannose was introduced after 10 minutes of ConA/copolymer incubation to obtain [α-D-mannose]=20 mM. To qualitatively assess time-dependent agglutination potential, the OD at 350 nm was plotted versus time.

### 3.8 Formation of Copolymer/pDNA Polyplexes and Lipoplexes

Polyplexes comprising the micellic assembly of triblock copolymers and pDNA were formed by combining equal volumes of pDNA (0.1 mg/ml in molecular biology grade water) and copolymer solutions (in Dulbecco's phosphate-buffered saline, pH 7.4 (PBS)) for 30 min at room temperature. Lipoplexes were formed by combining pDNA with Lipofectamine 2000 at a 3:1v/w L2K:DNA ratio in serum-free media in accordance with the manufacturer's protocol.

### 3.9 Gel Retardation Assay

The charge ratio (+/−) at which the triblock copolymer mediates complete pDNA condensation into a polyplex was determined via a gel retardation assay. The charge ratio is the molar ratio between protonated DMAEMA tertiary amines (assuming 50% protonation at physiological pH) and phosphate groups along the pDNA backbone. Triblock copolymer/pDNA polyplexes were formulated with 0.5 pg pDNA for 30 min followed by a 15 min incubation in the presence of FBS (final FBS concentration of 10 vol %). A 0.7% (w/v) agarose gel was loaded with each lane containing a separate treatment and subsequently run at 90V for one hour. The gels were stained with SYBR Gold prior to fluorescence visualization.

### 3.10 Dynamic Light Scattering (DLS)

The sizes of free triblock copolymer micelles and copolymer/pDNA polyplexes were determined by DLS measurements using a Malvern Zetasizer (Worcestershire, UK). Free copolymer measurements were performed at a polymer concentration of 100 μg/mL while triblock copolymer/pDNA polyplex particles were analyzed at a pDNA concentration of 5 μg/mL. All measurements were performed in the presence of 150 mM NaCl. Mean diameters are reported as the number average.

### 3.11 Hemolysis assay

The potential for the free triblock copolymer to disrupt endosomal membranes was assessed by a hemolysis assay according to standard protocols previously described in the art. Briefly, triblock copolymer was incubated in the presence of erythrocytes at 20 μg/mL in 100 mM sodium phosphate buffers (supplemented with 150 mM NaCl) of varying pH (7.4, 7.0, 6.6, 6.2, and 5.8) intended to mimic the acidifying pH gradient that endocytosed material is exposed to. The extent of cell lysis (i.e. hemolytic activity) was determined by detecting the amount of released hemoglobin via absorbance measurements at 492 nm.

### 3.12 Bone marrow-derived dendritic cell (BMDC) isolation

BMDCs were isolated from BALB/c mice using standard procedures. Briefly, bone marrow cells were collected from mouse femurs and tibias and treated with ACK lysis buffer to remove red blood cells. These cells were washed and cultured at 2×10⁶ cells/mL in 3 mL of complete RPMI media (2% HEPES buffer, 0.1 mM 2-mercaptoethanol, 100 U/mL penicillin, 100 μg/mL streptomycin, 2 mM glutamine, 10% fetal calf serum) supplemented with 10 ng/mL granulocyte-olony-stimulating factor (GM-CSF) and interleukin-4 (IL-4) (BD Biosciences, San Jose, Calif.) in six-well plates. By day 6 of this culture method, the cells were ready for treatment.

### 3.12 In Vitro Transfections

DC2.4 cells or BMDCs are seeded in 24-well plates in 1 ml complete medium (2×10⁵ cells/well in RPMI/10% FBS/antibiotics or in BDMC media, respectively) and cultured for 24 h to approximately 70% confluence. Micellic polyplexes and lipoplexes are formulated as described above; cells are washed once with PBS and incubated with polyplexes/lipoplexes at 1 μg DNA/well in 200 μl antibiotic-free medium for 4 h at 37° C. Cells are then washed and the medium replaced with 500 μL complete medium for an additional 44 h prior to analysis. Both lipoplexes and unmodified diblock copolymer (comprising the delivery and micelle-forming blocks) polyplexes can be selected as positive controls while free pDNA and PBS can be used as negative controls.

### 3.13 Flow Cytometry Analysis of Gene Expression

DC2.4 cells and BMDCs are incubated for 10 min at room temperature in PBS-based cell dissociation buffer (GIBCO, Carlsbad, Calif.) and collected by vigorous washing. Cell solutions are then added at approximately a 1:2 dilution to PBS containing 2% FBS and 0.2 μg/ml propidium iodide (PI, Invitrogen). GFP expression data can be acquired on a BD FACscan flow cytometer (BD Biosciences); 10,000 events gated on viable PI-negative cells will be collected per sample and analyzed in FlowJo (TreeStar, Ashland, Oreg.).

### 3.14 Lactate Dehydrogenase Cytotoxicity Assay

Cytotoxicity are evaluated by a lactate dehydrogenase (LDH) cytotoxicity detection kit (Roche). Cells are treated following the same protocol outlined above for transfections. Following the 44 h incubation, cells are washed, then lysed in 400 μL of RIPA Lysis Buffer (Pierce, Rockford, Ill.) at 4° C. for at least 1 h. Lysates are then diluted 2:3 in PBS in a 96-well plate (total volume 100 μL), combined with 100 μL of LDH substrate solution, incubated for 10-20 min at room temperature, and measured for absorbance at 490 nm (reference 650 nm). Cell viability are normalized to the PBS treatment control.

### 3.15 In Vitro pDNA Uptake Study

The same protocol for the in vitro transfections and flow cytometry GFP measurements can be followed to determine carrier-mediated uptake of pDNA with the following modifications. Cells are treated at the same concentrations for 2 h with Cy3-labeled pDNA. After this incubation, cells are washed, lifted accordingly, and diluted into PBS supplemented with 2% FBS 0.01% trypan blue (Invitrogen). The addition of trypan blue is intended to quench external (cell surface-associated) fluorescence. Flow cytometry collection and analysis proceed as detailed previously.

### 4. Results and Discussion

### 4.1 Triblock Copolymer Synthesis and Characterization

To prepare the mannosylated triblock copolymer, an acetylated glycomonomer was used due to the organic solvent conditions employed in the nontargeted diblock copolymer synthesis (Scheme 4). The successful RAFT-mediated synthesis of this triblock copolymer, p(AcManEMA-b-DMAEMA-b-[DEAEMA-co-BMA]), was confirmed via ¹H-NMR and GPC (see Table 2), and resulted in a final triblock copolymer with a narrow polydispersity (PDI=1.15) and a BMA composition of 45 mol % in the third block. 40% BMA for this block was shown to be optimal in the transfection of RAW 264.7 cells for the diblock copolymer. The acetylated triblock copolymer was deprotected to yield pendant mannose residues in the native D-conformation via a base-talyzed saponification reaction (Scheme 5). The removal of the acetyl groups was validated by ¹H-NMR (not shown) as resonances associated with these protecting moieties (δ=˜2.2-2.0) were no longer present in the deprotected polymer while the remaining pendant functionalities were retained.

TABLE 2
Molecular weights^{a}, polydispersities^{a}, conversions^{b} and
compositions^{b} for protected triblock copolymer.

| | Mₙ | Mₙ | | | |
|---|---|---|---|---|---|
| | AcMannose | DMAEMA | | | |
| | Block^{c} | Block^{d} | Mₙ EB40 | Total Mₙ | |
| Polymer | (g/mol) | (g/mol) | Block^{e} (g/mol) | (g/mol) | PDI |
| pAcMan | 11100 | — | — | 11100 | 1.09 |
| Diblock | 11100 | 5100 | — | 16200 | 1.02 |
| Triblock | 11100 | 5100 | 14200 | 30400 | 1.15 |

^{a}As determined by GPC
^{b}As determined by ¹H-NMR
^{c}71% monomer conversion
^{d}52% monomer conversion
^{e}Experimental 45% BMA; theoretical 40% BMA

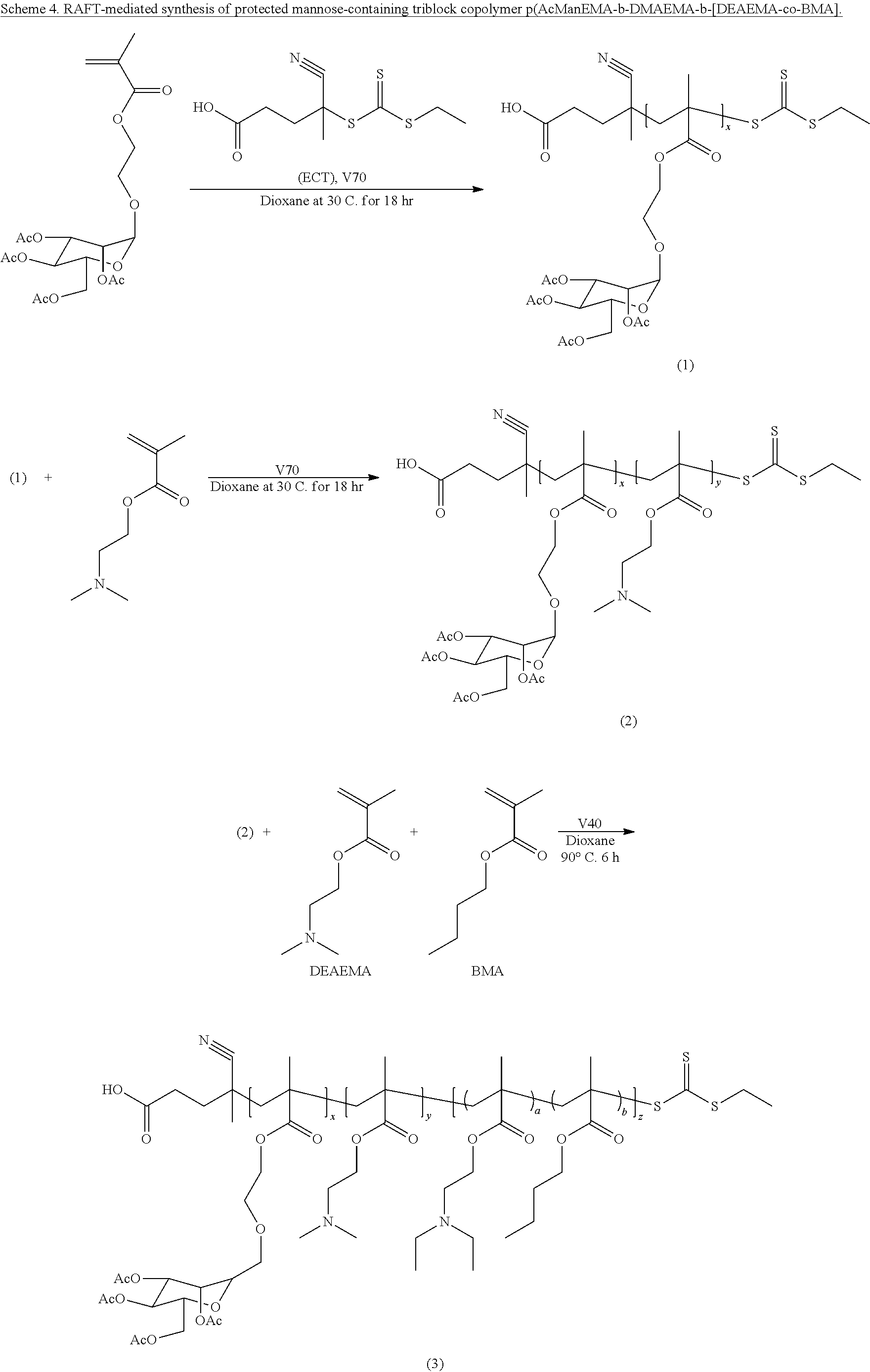

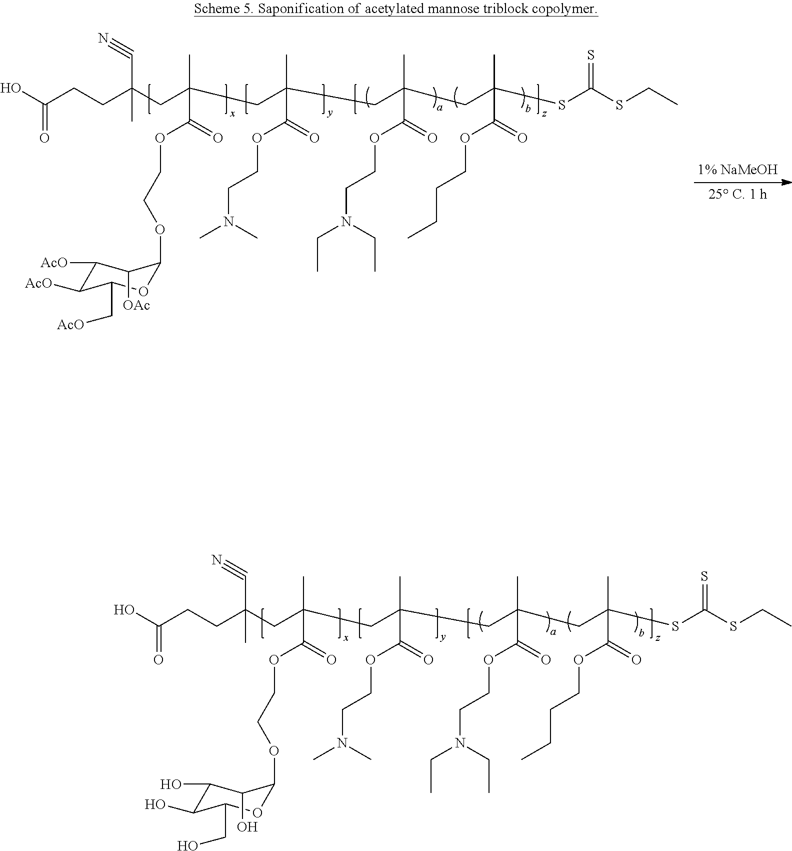

### 4.2 Evaluation of Triblock Copolymer pDNA-Condensation Ability

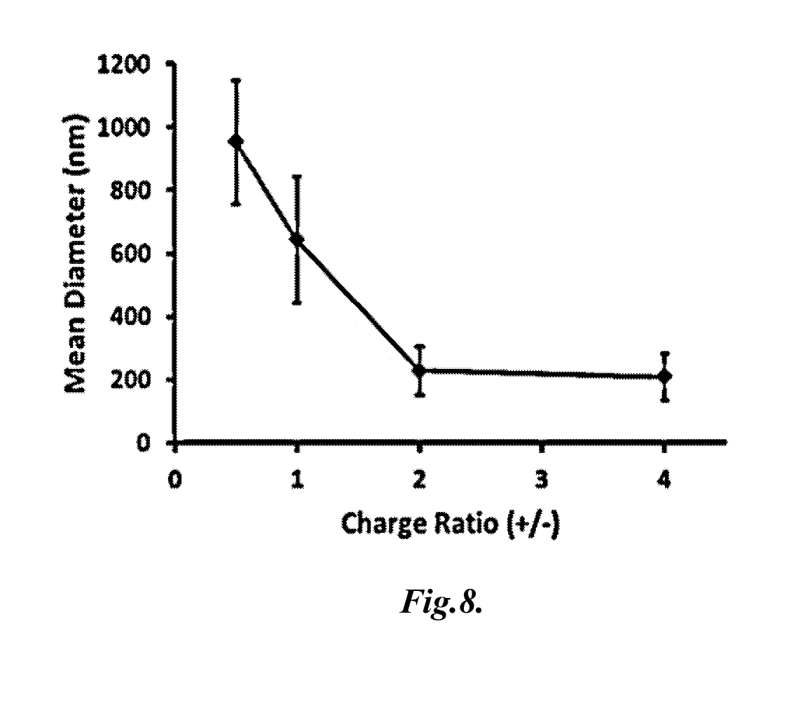

Polyplexes between the micellic assemblies formed by the triblock copolymers and pDNA were prepared to determine the charge ratio (+/−) necessary to condense the nucleic acid into stable particles. The copolymer was able to completely prevent migration of pDNA at a charge ratio of 1 as observed by a gel retardation assay (data not shown). At a charge ratio of 2, the resultant polyplexes were 230±80 nm (FIG. 8). These sizes were similar to what has been observed for the unmodified diblock copolymers comprising the micelle-forming, pH-responsive endosomolytic block and a functional delivery block.

### 4.3 Determination of pH-Responsive Behavior of Free Triblock Copolymer Micelles and Polyplexes

The unique pH-responsive endosomolytic behavior of these micelles depends on a conformational shift from a membrane-inert particle to a membrane-interactive unimer. For the triblock copolymer, this structure-property relationship was validated via DLS and hemolysis across a range of endosomal-lysosomal relevant pH values (FIGS. 9 and 10). At pH 6.6, indicative of a late endosome, particles were no longer observed via DLS and hemolytic activity was observed; this activity became more prominent below pH 6.6. These two experiments validate retention of the pH-dependent structure-activity relationship for the triblock copolymer.

Electrostatic complexes between the described triblock copolymers of the present invention and pDNA have yet to be determined. However, previously generated data from the diblock copolymer (comprising the micelle-forming, pH-responsive endosomolytic block and a functional delivery block) demonstrated an increase in polyplex size as pH decreased, suggestive that the unimer copolymer conformation is unable to condense pDNA as effectively as the micelle conformation due to a decrease in the relative polycation charge density despite an increase in amine protonation. Hemolysis of the polyplexes formed by these diblock copolymers demonstrated similar hemolytic trends with little reduction in overall activity (data not shown).

### 4.4 Determination of Mannose-Mediated Lectin-Binding by Triblock Copolymer

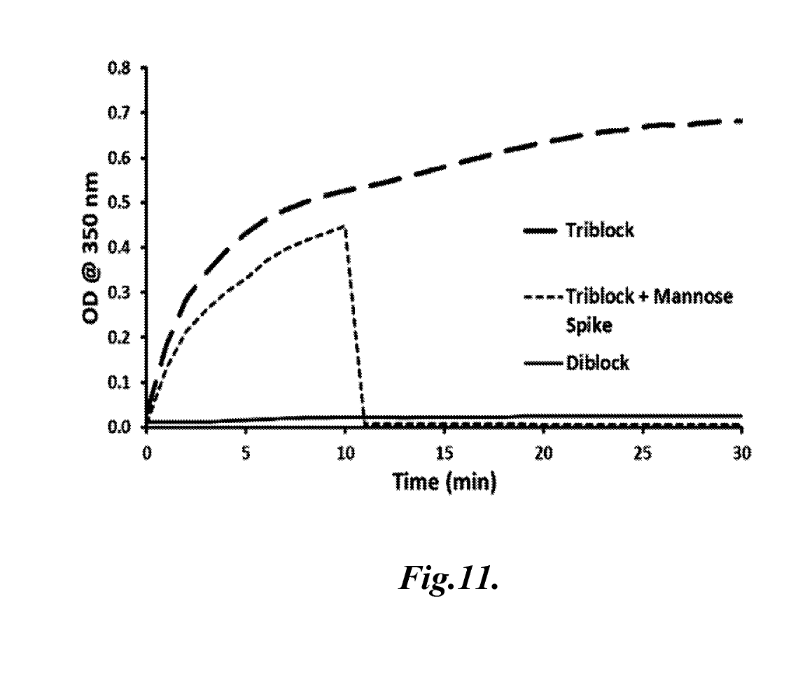

An agglutination assay with a mannose-specific lectin, Concanavalin A (ConA), was performed to evaluate whether the mannose-containing triblock copolymer is capable of engaging mannose-binding CRDs (FIG. 11). Upon addition of the mannose-containing triblock, a time-dependent increase in turbidity was observed likely due to mannose-mediated occupation of ConA CRDs cross-bridging the tetrameric protein. Adding free α-D-mannose resulted in abrogation of this aggregation behavior. Along with this finding and the lack of measurable increases in turbidity from a related diblock copolymer, the presence of mannose on the triblock copolymer capable of engaging in multivalent lectin-interactions was validated.

### 4.5 Evaluation of In Vitro Transfection Efficacy and Toxicity of Targeted and Nontargeted Copolymer Micelles

To test whether carbohydrate-mediated targeting enhanced DNA transfection, dendritic cell types are selected which are known to express MMR, a receptor that specifically recognizes and internalizes mannose. Exemplary cell types include: DC2.4 cells (murine dendritic immortalized cell line) and BMDCs (murine bone marrow-derived dendritic cells). Transfection efficacy and toxicity of targeted (e.g., triblock) and nontargeted (e.g., diblock containing a micelle-forming, pH-responsive endosomolytic block with a functional delivery block) copolymer micelles are assayed, for example, as described above. Although the nontargeted diblock copolymer has demonstrated the ability to internalize into macrophage- and dendritic-like cell lines via nonspecific mechanisms, it is expected that the addition of active targeting will enhance uptake, and therefore transfection activity. This expectation is based upon previous findings that have modified polycation-based carriers with carbohydrates and observed an increase in transfection activity.

Additional assays to characterize and optimize transfection efficacy and reduce cytotoxicity can be performed. For example, heparin and serum-protein displacement assays could be performed comparing the triblock and nontargeted diblock copolymers to investigate whether the mannose affects the ability of the pDNA to dissociate from the polyplex. Moreover, the role of the mannose block in the pH-dependent structure-activity relationship of the resultant polyplex can be explored by performing DLS and hemolysis assays on the polyplexes and comparing the data between the triblock and nontargeted diblock copolymers. Alternative synthetic strategies can also be explored and are described below.

### 4.6 Determination of In Vitro Carbohydrate-Mediated pDNA Uptake Specificity

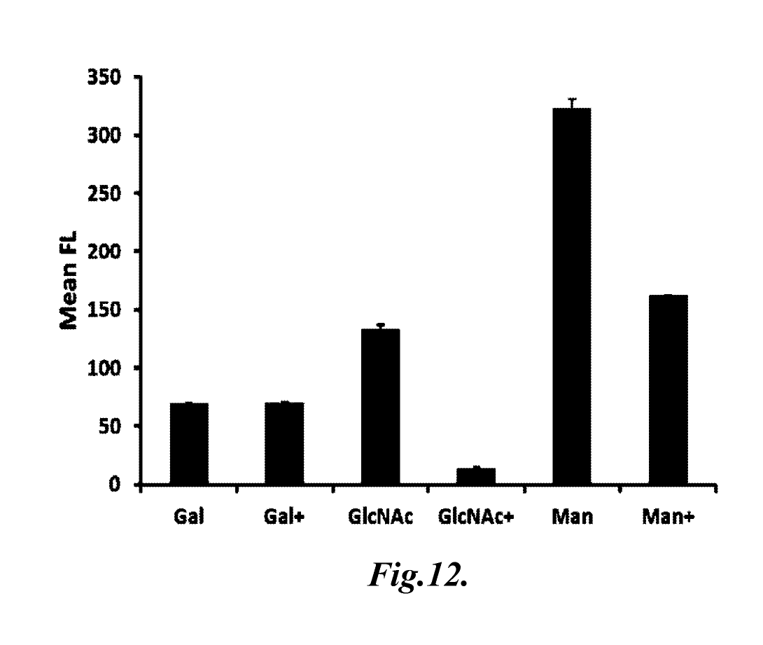

The ability of the mannosylated triblock copolymer to mediate mannose-specific cellular uptake can be evaluated in target cell types using flow cytometry after treatment with complexed Cy3-labeled pDNA. The same rationale for observing enhanced transfection activity applies to the expected results for these experiments. It is noted that preliminary studies demonstrated that the linear mannose-containing copolymers prepared as described above exhibit the same mannose specificity in BMDCs as they do in BMDMs (FIG. 12), suggesting that a similar, if not the same, C-type lectin is facilitating uptake.

Potential factors influencing the specific uptake of the mannosylated triblock can be optimized. For example, the incubation period for the micellic assembly/pDNA polyplex uptake may be a more important parameter to consider than for the linear carbohydrate-containing copolymers because antigen-presenting cells are highly phagocytic and nonspecifically internalize particles. Additionally, the presence of polycations on the carrier may facilitate electrostatic-mediated adsorptive uptake in vitro. Receptor-mediated uptake may, therefore, only be observed at shorter incubation times as nonspecific means of uptake are likely to dominate over time. Thus, treatment parameters (e.g. incubation time, pDNA concentration, and polyplex charge ratio) can be optimized to ascertain whether mannosylation effects uptake specificity of the polyplex in vitro.

### 5. Conclusion

A mannosylated triblock copolymer was successfully synthesized by adapting a new copolymer synthetic scheme to previous conditions established for the nontargeted diblock copolymers. Characterization assays indicated that triblock co-polymers assembled into micelles, and that stable complexes formed between the triblock copolymer micelles and plasmid DNA, thus confirming the ability of the triblock polymers to condense pDNA. Additionally, the free triblock copolymer micelles exhibited pH-responsive disassociation resulting in membrane-interactive unimers, indicating that the mannosylated triblock polymers are likely to retain the endosomolytic properties previously established for the nontargeted diblock polymers. The triblock copolymers also retained mannose-mediated lectin binding properties demonstrated above for the mannose-containing copolymers. In combination, these results demonstrate the applicability of the triblock copolymer as a cell-specific delivery system for biologic compositions.

Further assays directed to optimization of cell-specific transfection efficiency, optimization of carbohydrate-mediated uptake specificity and reduction in target cell cytotoxicity are proposed. These approaches are outlined in the previous section and consist of two primary strategies: modification of blocking order and block length for the triblock copolymer scheme and the formation of multifunctional ternary complexes.

### In Vivo Stimulation of Immune Response by Copolymer Micelles Delivering Plasmid DNA Encoding Polyepitope Cancer Epitopes

### 1. Rationale and Overview:

Both targeted (e.g., triblock) and nontargeted (e.g., diblock comprising the micelle-forming, pH-responsive endosomolytic block and a functional delivery block) copolymer micelles prepared as described in the above embodiments can be examined as carriers of therapeutically relevant pDNA in an in vivo murine model. Comparisons between the complexed and free pDNA treatments can provide a measure of carrier efficacy while differences between the two classes of carriers can offer insight into the importance of mannose targeting in vaccine therapies. For example, pDNA encoding one or multiple polyepitope of the insulin-like growth factor 1 receptor (IGF-IR), a biomarker over-expressed in an ovarian tumor microenvironment, can be condensed by the two classes of carriers and the resultant complexes is characterized. Intradermal administration of these complexes (with co-administered adjuvants) is performed and the resulting immune responses is measured by a combination of intracellular cytokine (ICC) staining and enzyme-linked immunosorbent spot assay (ELISPOT) to determine antigen-specific CD4+ and CD8+ T cell activation and indirect enzyme-linked immunosorbent assay (ELISA) to determine antibody titers.

### 2. Introduction

Epithelial ovarian cancer is an aggressive malignancy often diagnosed late in the progression of the disease, making conventional treatments ineffective at preventing recurrence. The disease is characterized by neoplasia of the ovarian epithelium whereby healthy cell growth is perturbed and uncontrolled cell proliferation develops. Numerous factors have been implicated in the onset of this cancer including the lack of transcriptional control over the proteins in the insulin-like growth factor (IGF) regulatory network. The functions of these proteins are related to metabolism and cell growth with relative expression levels dictated by positive and negative feedback mechanisms. Overexpression of a protein with positive feedback activities (e.g. the IGF-I receptor (IGF-IR)) can induce malignancy in healthy tissue. Elevated levels of IGF-IR have been observed in epithelial ovarian cancer, suggesting that exploitation of this pathway is one mechanism by which the cancer develops.

Tumorigenesis, the process by which malignant cells develop into tumors, can occur unhindered due to the way in which cancerous cells are able to subvert the body's own immune system. Under homeostatic conditions, immune cells can recognize and eliminate malignant cells by exerting specific effector functions. The tumor microenvironment is characterized as being immunosuppressive and, therefore, interferes with these immune surveillance activities. Cancer immunotherapy intends to overcome this suppression by directing a potent immune response to the tumor site. For example, it has been previously reported that patients with epithelial ovarian cancer who were able to endogenously generate a T cell response within the tumor had a higher survival rate over a five-year period compared to those without the intratumoral T cell population (34% vs. 4.5%). In light of this study, therapeutic strategies which can direct anti-tumor T cells to the site of malignancy have generated interest in treating ovarian cancer.

Cancer vaccines provide an attractive route to elicit anti-tumor immunity. This approach involves the delivery of tumor-associated antigenic material (proteins, peptides, or antigen-encoding DNA) to antigen-presenting cells (APCs). These cells process the antigens into peptide fragments that associate with major histocompatibility complexes (MHCs) that are presented on the cell surface. When the MHC-peptide ensembles are recognized by T cells, along with appropriate co-stimulatory signals provided by APCs, the cells become activated towards the specific antigen and their associated effector function is engaged. Activated CD8+ T cells become cytotoxic T lymphocytes (CTLs) that can directly lyse cells expressing the antigen. In the tumor, this lytic activity is suppressed due to the aforementioned tumor evasion mechanisms. One approach to tip the balance to tumor cell destruction is to specifically activate CD4+ T helper (Th) cells with a Th1 phenotype. In addition to directing the proliferation of CTLs, these cells can modulate the immune profile of the tumor microenvironment by secreting cytokines that upregulate endogenous antigen presentation and recruit innate immune cells. Eliciting tumor antigen-specific Th1 cells is, therefore, a potent strategy for developing cancer vaccines.

Described herein is an embodiment for the in vivo evaluation of copolymer carriers delivering DNA encoding one or more cancer-specific antigens. DNA encoding polyepitope are capable of generating a broad (i.e. multiple-epitope recognizing) response. The copolymer carriers are intended to increase transfection of the DNA by APCs, thereby enhancing the amount of antigen being presented. Additionally, the role of mannose-mediated targeting on improving delivery to Langerhans cells (LCs), a subset of dendritic cells (DCs) located in the skin that are potent mediators of immune responses can be investigated. The LCs express C-type lectins that specifically recognize and internalize mannose. Granulocyte-macrophage colony-stimulating factor (GM-CSF) can be used as an adjuvant due to its ability to mobilize LCs to the site of administration and elicit DNA-mediated Th1 immune responses. This proposed study evaluates whether copolymer carriers can improve the efficacy of DNA-based vaccines in an in vivo murine model by examining the proliferation of antigen-specific effector T cell populations, a parameter that correlates with positive clinical responses.

### 3. Materials and Methods

### 3.1 Materials

Most chemicals and materials are available from Sigma-Aldrich (St Louis, Mo.), unless otherwise specified. pUMVC3 is amplified in E. coli followed by purification with Qiagen EndoFree Plasmid Maxi Kit (Qiagen Inc., Valencia, Calif.). pDNA stocks are prepared in deionized water and stored at −20° C. Nontargeted diblock and mannosylated triblock copolymers can be prepared following procedures described herein above. Antibodies are available from BD Biosciences (San Jose, Calif.), unless otherwise specified. Custom peptide sequences are available from GenScript (Piscataway, N.J.).

### 3.2 Mice

Female BALB/c mice, 6 to 8 weeks old, are maintained as described above.

### 3.3 Formation of copolymer/05NA polyplexes

Copolymer/pDNA polyplexes are formed by combining equal volumes of pDNA (0.1 mg/ml in molecular biology grade water) and copolymer solutions (in Dulbecco's phosphate-buffered saline, pH 7.4 (DPBS)) for 30 min at room temperature.

### 3.4 Copolymer/pDNA Polyplex Characterization

Copolymers are evaluated for the ability to condense the pDNA into nanoparticles as a function of charge ratio (+/−). Gel retardation assays are performed in the presence of serum to demonstrate particle stability. Briefly, following the formation of polyplexes, fetal bovine serum (FBS) are added to the solutions (final FBS concentration of 10 vol %) and allowed to incubate for 15 min. A 0.7% (w/v) agarose gel are loaded with each lane containing a separate treatment and subsequently run at 90V for one hour. The gels are stained with SYBR Gold prior to fluorescence visualization. Particle size are measured by dynamic light scattering (DLS) using a Malvern Zetasizer (Worcestershire, UK) at a pDNA concentration of 5 μg/mL in PBS. Mean diameters are reported as the number average.

### 3.5 Determination of Copolymer/pDNA Polyplex pH-Responsive Activity

To ensure that the copolymers maintain pH-responsive activity following pDNA condensation, a pH-dependent DLS study and hemolysis assay can be performed. The first experiment follows the same DLS protocol as provided above, except the charge ratio is fixed and five PBS (0.1M sodium phosphate and 0.15 M NaCl) buffers of varying pH (7.4, 7.0, 6.6, 6.2, and 5.8) are used. A hemolysis assay uses these same buffers to assess the potential for the polyplexes to disrupt endosomal membranes. The protocol suggested here has been described previously. Briefly, polyplexes are incubated in the presence of erythrocytes at 20 μg/mL in 100 mM sodium phosphate buffers (supplemented with 150 mM NaCl) of varying pH intended to mimic the acidifying pH gradient that endocytosed material is exposed to. The extent of cell lysis (i.e. hemolytic activity) is determined by detecting the amount of released hemoglobin via absorbance measurements at 492 nm.

### 3.6 Immunization of Mice

The following treatments can be evaluated for immune responses: free pDNA, diblock copolymer/pDNA polyplexes (nontargeted) and mannosylated triblock copolymer/pDNA polyplexes (targeted) in addition to a PBS control. For the polyplexes, the charge ratios to be selected are based on the minimum amount of copolymer necessary to form stable active polyplexes, likely +/−2 or 4 if consistent with the studies described above. Following an established murine vaccination protocol, six mice per group are administered the treatments once every two weeks for six weeks at 30 μg pDNA diluted to 50 μL total volume with 1×DPBS. Polyplexes are formulated under the same conditions described above at concentrations necessary to achieve the final administered concentration. All samples, including PBS, are supplemented with GM-CSF as an adjuvant. Mice are injected intradermally (i.d.) in the pinna of the ear. One week after the final injections, mice are sacrificed and sera and spleens will be harvested to assess immune responses. Splenocytes are isolated using standard protocols. Briefly, spleens are pressed through a wire strainer, washed and then incubated with ACK Lysis Buffer (Invitrogen, Carlsbad, Calif.) at 37° C. for 5 min to lyse red blood cells. The cell solution are then washed, counted, and then resuspended in mouse T cell media (RPMI 1640 supplemented with 10% FBS, 1% penicillin-streptomycin, and 10 mM 2-mercaptoethanol) to a final concentration of 3×10⁶ cells/mL.

### 3.7 Enzyme-Linked Immunosorbent Spot Assay (ELISPOT)

To determine the frequency of IFN-γ positive splenocytes in each group, a modified 10-day ELISPOT assay can be employed. Splenocytes are added to a 96-well plate at a density of 3×10⁶ cells/well in 100 μL of the previously-described T cell media. To each spleen, either tetanus toxoid (at 525 LFU/mL) or a mixture of the five peptides (at 100 μg/mL) are added in triplicate in addition to an unstimulated control, bringing the total cell volume to 200 μL. The cells are allowed to incubate at 37° C. for 4 days in the presence or absence of antigenic stimulation. Next, 20 μL of 100 U/mL recombinant interleukin-2 (rIL-2) are added to each well and the cells are incubated for an additional 3 days. In parallel, a 96-well nitrocellulose (NC)-backed plate (MAIP S4510; Millipore, Moisheim, France) of immobilized anti-mouse IFN-γ′ antibody (Mabtech, Stockholm, Sweden) is prepared by incubating the antibody overnight at 4° C. (10 μg/mL diluted in 1×PBS to 50 μL/well). The NC plate is washed 3× with PBS and then to it, 200 μL/well of 2% bovine serum albumin (BSA) in 1×PBS (blocking buffer) is added and allowed to incubate for 2 hours at 37° C. After washing 3× with PBS, the NC plate is prepared for subsequent cell culture. The incubating splenocytes is then removed from the incubator, centrifuged, and resuspended with the same concentrations of the previous antigen treatments in addition to irradiated autologous splenocytes (at 3×10⁵ total cells per well). The entire contents from each well on the cell culture plate are then transferred to a corresponding well on the NC plate. The NC plate is incubated at 37° C. for an additional two days. The plate is then removed and washed 1× with PBS and 2× with 0.05% Tween-20 in PBS. Biotinylated anti-mouse IFN-γ antibody at 5 μg/mL is added to each well at a total volume of 50 μL and the plate is incubated overnight at 4° C. The plate is then washed again and streptavidin-alkaline phosphatase (AP:diluted 1:1000 in PBS) is added to each well (50 μL total). Following a two-hour incubation, the plates are washed and subsequently incubated in the presence of an APcalorimetric substrate (Bio-Rad, Hercules, Calif.) for 20-30 minutes to allow for color development. The reaction is then halted by rinsing the plate with cold distilled water (at least 4×). After the plate has been allowed to thoroughly dry, spots are visualized and counted on an AID ELISPOT reader (Autoimmun Diagnastika GmbH, Strassberg, Germany). Data is reported as mean spot forming units (s.f.u.) per 3×10⁵ cells±standard error of the mean (SEM).

### 3.8 Intracellular Cytokine (ICC) Staining and Flow Cytometry

Splenocytes can be isolated as described previously and characterized using the following modified protocol. Briefly, splenocytes are cultured on a 96-well plate and treated with and without antigenic stimulation from the peptide mixture (100 μg/mL) for six hours with 2 M monensin (GolgiStop; BD Biosciences, San Jose, Calif.). The cells are then washed and stained with either FITC-anti-CD4 or FITC-anti-CD8 monoclonal antibody (mAb). Following an additional wash, the cells are fixed with paraformaldehyde in the presence of a permeabilization agent, saponin, and stained with PE-anti-IFN-7 mAb. Cells are washed a final time and suspended in PBS supplemented with 2% FBS, counted using a FACSCanto (BD Biosciences), and analyzed in FlowJo (TreeStar).

### 3.9 Statistical Analysis

ANOVA can be used to test for treatment effects at a significance of p<0.05, and Tukey's test can be used for post hoc pairwise comparisons between individual treatment groups.

### 4. Expected Results

### 4.1. Copolymer/pDNA Polyplex Characterization

The capacity for both targeted and nontargeted copolymers to condense the pDNA into serum-stable nanoparticles is expected based on previous studies investigating similar pUC-derived pDNA complexation. Additionally, the pH-responsive behavior of the triblock copolymers (as measured by DLS and hemolytic assays) is expected to be maintained after polyplex formation based on the characterization results provided in the embodiment described above. As long as hemolytic activity is retained at low pH values (≦ 6.6, indicative of late endosomal/lysosomal compartments) and the formed polyplexes are on the nm-size scale (≦ ˜200 nm, the approximate limit for nonspecific macropinocytosis), the polyplexes are available for assays addressing in vivo transfection efficacy.

### 4.2 T-Cell Mediated Immune Response

Both ELISPOT and ICC staining/flow cytometry offer complementary analytical tools to probe for cell-mediated immune responses directed by T-cells. ELISPOT can provide data pertaining to the relative frequency of antigen-specific, IFN-γ positive cells among the heterogeneous milieu of splenocytes. However, the assayed population does not discriminate between CD4+ and CD8+ positive T cells and could potentially include additional IFN-γ-secreting lymphocytes. Studies examining DNA vaccine-generated immune responses have observed anywhere from ˜0.02-0.1% IFN-γ+ cells for a mannan-targeted OVA construct administered i.d. to ˜0.1% for a polyepitope vaccine lacking a carrier administered subcutaneously.

The relative percentage of antigen-specific lymphocytes is expected to appear higher following ICC staining/flow cytometry due to the initial gating with either CD4+/CD8+ selection effectively “enriching” the analyzed population. Additionally, this gating procedure permits discrimination between primed CD8+ and CD4+ Th1 cells. Immunizations with HER/neu-based DNA vaccines have produced ˜1% CD8+IFN-γ+ cells in studies examining different adjuvant co-delivery strategies. However, in the previous studies, no carrier was utilized and only naked pDNA was administered. Due to the enhanced transfection efficacy anticipated from the copolymer micelle carriers, as a result of DC-specific targeting for the mannosylated copolymer and the endosomolytic properties of each copolymer, equal or better T cell responses are expected.

### 4.3 Antibody-Mediated Immune Response

Based on previous findings that DNA vaccines bias T helper cells towards a Th1 phenotype, high populations of neutralizing antibodies as mediated by Th2 cells are not expected. Th1 cells, however, are capable of eliciting the production of opsonizing antibodies. Therefore, antigen-specific antibodies should be present at high enough levels in the sera to detect via ELISA if pDNA transfection is successful. A significant antibody response can be attributed to samples that deviate from the PBS control by at least two standard deviations.

### 5. Conclusion

The goal of these studies is to both validate the copolymer materials as efficacious pDNA carriers for therapeutically relevant vectors and to evaluate the contribution of mannose targeting in i.d. administration of complexed pDNA. As such, the immune response elicited by the copolymer/pDNA carriers serves as the endpoint readout as it addresses these two key criteria. A comparison of polyplexes to naked pDNA can demonstrate elevated transfection activity by the carrier and a comparison of targeted to nontargeted copolymers can illustrate the importance of localizing pDNA to DCs for immunization. Additional studies can also examine epitope-specific T cell populations by restimulating with individual peptides prior to ELISPOT and ICC to determine which sequences are predominantly activating effector cells. Upon demonstration that the copolymers mediate potent immune responses, a therapeutic immunization study can be evaluated in a therapeutic model of disease. The ability of the copolymer/pDNA polyplexes to elicit anti-tumor immunity would be evaluated in these studies, as measured by inhibition of tumor growth. These experiments allow for epitope-spreading to be examined, a phenomena that is associated with more positive clinical outcomes following immunotherapy.

While illustrative embodiments have been illustrated and described, it will be appreciated that various changes can be made therein without departing from the spirit and scope of the invention. Aspects of the disclosure can be modified, if necessary, to employ the systems, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. Accordingly, the disclosure is not limited.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

All of the references cited herein are hereby incorporated by reference in their entirety.

1. A block copolymer, comprising:
(a) a first block comprising repeating units having pendant carbohydrate groups; and
(b) a second block comprising repeating units having pendant functional groups suitable for associating a therapeutic agent and/or a diagnostic agent to the block copolymer.

2. The copolymer of claim 1 further comprising a third block coupled to the terminus of the second block, the third block comprising repeating units having membrane destabilizing functionality.

3. The copolymer of claim 1, wherein the first block further comprises repeating units having neutral pendant groups.

4. The copolymer of claim 2, wherein the first block further comprises repeating units having neutral pendant groups.

5. A copolymer, comprising:
(a) repeating units having pendant carbohydrate groups; and
(b) repeating units having pendant functional groups suitable for associating a therapeutic agent and/or a diagnostic agent to the block copolymer.

6. The copolymer of claim 5 further comprises repeating units having neutral pendant groups.

7. A block copolymer, comprising:
(a) a first block comprising repeating units having membrane destabilizing functionality; and
(b) a second block comprising
(i) repeating units having pendant carbohydrate groups; and
(ii) repeating units having pendant functional groups suitable for associating a therapeutic agent and/or a diagnostic agent to the block copolymer.

8. The copolymer of claim 7, wherein the second block further comprises repeating units having neutral pendant groups.

9. The copolymer of claim 1 further comprising an associated therapeutic agent and/or diagnostic agent.

10. The copolymer of claim 9, wherein the therapeutic agent is selected from the group consisting of a protein, peptide, and oligonucleotide.

11. The copolymer of claim 9, wherein the diagnostic agent is selected from the group consisting of a fluorescent agent, a magnetic resonance imaging agent, and a radiolabel.

12. The copolymer of claim 1, wherein the carbohydrate is selected from the group consisting of mannose, galactose, fucose, N-acetyl glucosamine, sialic acid, and combinations thereof.

13. The copolymer of claim 1, wherein the repeating units having pendant functional groups suitable for associating a therapeutic agent and/or diagnostic agent to the copolymer have pendant thiol or disulfide groups.

14. The copolymer of claim 1, wherein the repeating units having pendant functional groups suitable for associating a therapeutic agent to the block copolymer have pendant amine or cationic groups.

15. The copolymer of claim 2, wherein the repeating units having membrane destabilizing functionality comprise repeating units having carboxylic acid groups.

16. The copolymer of claim 15, wherein the repeating units having pendant carboxylic acid groups are selected from the group consisting of acrylic acid repeating units, C1-C8 alkyl acrylic acid repeating units, and mixtures thereof.

17. The copolymer of claim 2, wherein the third block further comprises repeating units having neutral pendant groups.

18. The copolymer of claim 17, wherein the repeating units having neutral pendant groups are selected from C1-C8 alkyl acrylic acid C1-C8 ester repeating units, acrylic acid C1-C8 ester repeating units, and mixtures thereof.

19. The copolymer of claim 2, wherein the third block further comprises repeating units having pendant amine groups.

20. The copolymer of claim 19, wherein the third block comprises a substantially equimolar amount of repeating units having pendant carboxylic acid groups and repeating units having pendant amine groups.

21. The copolymer of claim 7, wherein the repeating units having membrane destabilizing functionality comprise repeating units having carboxylic acid groups.

22. The copolymer of claim 21, wherein the repeating units having pendant carboxylic acid groups are selected from the group consisting of acrylic acid repeating units, C1-C8 alkyl acrylic acid repeating units, and mixtures thereof.

23. The copolymer of claim 7, wherein the first block further comprises repeating units having neutral pendant groups.

24. The copolymer of claim 23, wherein the repeating units having neutral pendant groups are selected from C1-C8 alkyl acrylic acid C1-C8 ester repeating units, acrylic acid C1-C8 ester repeating units, and mixtures thereof.

25. The copolymer of claim 7, wherein the first block further comprises repeating units having pendant amine groups.

26. The copolymer of claim 25, wherein the first block comprises a substantially equimolar amount of repeating units having pendant carboxylic acid groups and repeating units having pendant amine groups.

27. The copolymer of claim 1 having formula (I): 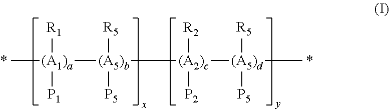
wherein
A₁(R₁)(P₁) is a first block repeating unit, wherein A₁ is the backbone of the repeating unit for the first block, R₁ is substituent of the repeating unit selected from the group consisting of hydrogen and C1-C8 alkyl optionally substituted with one or more fluorine atoms, and P₁ is a pendant group comprising a carbohydrate,
A₂(R₂)(P₂) is a second block repeating unit, wherein A₂ is the backbone of the repeating unit for the second block, R₂ is substituent of the repeating unit selected from the group consisting of hydrogen and C1-C8 alkyl optionally substituted with one or more fluorine atoms, and P₂ is a pendant group comprising a functional group suitable for associating a therapeutic agent to the block copolymer,
A₅(R₅)(P₅) is a repeating unit, wherein A₅ is a backbone of the repeating unit, R₅ is substituent of the repeating unit selected from the group consisting of hydrogen and C1-C8 alkyl optionally substituted with one or more fluorine atoms, and P₅ is a pendant group comprising a neutral group,
a is the mole fraction of the repeating unit in the first block, from 0.1 to 1.0,
b is the mole fraction of the repeating unit in the first block, from 0.0 to 0.99,
c is the mole fraction of the repeating unit in the second block, from 0.1 to 1.0,
d is the mole fraction of the repeating unit in the second block, from 0.0 to 0.99,
x is the mole fraction of the first block, from 0.01 to about 0.99, and
y is the mole fraction of the second block, from 0.01 to about 0.99.

28. The copolymer of claim 2 having formula (II): 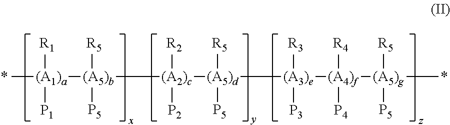
wherein
A₁(R₁)(P₁) is a first block repeating unit, wherein A₁ is the backbone of the repeating unit for the first block, R₁ is substituent of the repeating unit selected from the group consisting of hydrogen and C1-C8 alkyl optionally substituted with one or more fluorine atoms, and P₁ is a pendant group comprising a carbohydrate,
A₂(R₂)(P₂) is a second block repeating unit, A₂ is the backbone of the repeating unit for the second block, R₂ is substituent of the repeating unit selected from the group consisting of hydrogen and C1-C8 alkyl optionally substituted with one or more fluorine atoms, and P₂ is a pendant group comprising a functional group suitable for associating a therapeutic agent to the block copolymer,
A₃(R₃)(P₃) is a third block repeating unit, wherein A₃ is a backbone of the repeating unit for the third block, R₃ is substituent of the repeating unit selected from the group consisting of hydrogen and C1-C8 alkyl optionally substituted with one or more fluorine atoms, and P₃ is a pendant group comprising a carboxylic acid,
A₄(R₄)(P₄) is a third block repeating unit, wherein A₄ is a backbone of the repeating unit for the third block, R₄ is substituent of the repeating unit selected from the group consisting of hydrogen and C1-C8 alkyl optionally substituted with one or more fluorine atoms, and P₄ is a pendant group comprising an amine,
A₅(R₅)(P₅) is a repeating unit, wherein A₅ is a backbone of the repeating unit for the repeating unit, R₅ is substituent of the repeating unit selected from the group consisting of hydrogen and C1-C8 alkyl optionally substituted with one or more fluorine atoms, and P₅ is a pendant group comprising a neutral group,
a is the mole fraction of the repeating unit in the first block, from 0.01 to 1.0,
b is the mole fraction of the repeating unit in the first block, from 0.0 to 0.99,
c is the mole fraction of the repeating unit in the second block, from 0.01 to 1.0,
d is the mole fraction of the repeating unit in the second block, from 0.0 to 0.99,
e is the mole fraction of the repeating unit in the third block, from 0.0 to 1.0,
f is the mole fraction of the repeating unit in the third block, from 0.0 to 1.0, wherein e+f is at least 0.01,
g is the mole fraction of the repeating unit in the third block, from 0.0 to 0.99,
x is the mole fraction of the first block, from 0.01 to about 0.98,
y is the mole fraction of the second block, from 0.01 to about 0.98, and
z is the mole fraction of the third block, from 0.01 to about 0.98.

29. The copolymer of claim 5 having formula (III): 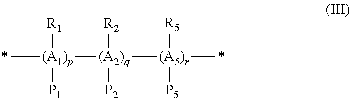
wherein
A₁(R₁)(P₁) is a first repeating unit, wherein A₁ is the backbone of the repeating unit, R₁ is substituent of the repeating unit selected from the group consisting of hydrogen and C1-C8 alkyl optionally substituted with one or more fluorine atoms, and P₁ is a pendant group comprising a carbohydrate,
A₂(R₂)(P₂) is a second repeating unit, wherein A₂ is a backbone of the repeating unit, R₂ is substituent of the repeating unit selected from the group consisting of hydrogen and C1-C8 alkyl optionally substituted with one or more fluorine atoms, and P₂ is a pendant group comprising a functional group suitable for associating a therapeutic agent to the block copolymer,
A₅(R₅)(P₅) is a third repeating unit, wherein A₅ is a backbone of the repeating unit, R₃ is substituent of the repeating unit selected from the group consisting of hydrogen and C1-C8 alkyl optionally substituted with one or more fluorine atoms, and P₅ is a neutral pendant group,
p is the mole fraction of the repeating unit, from 0.01 to 99,
q is the mole fraction of the repeating unit, from 0.01 to 0.99, and
r is the mole fraction of the repeating unit in the second block, from 0.0 to 0.99.

30. The copolymer of claim 7 having formula (IV): 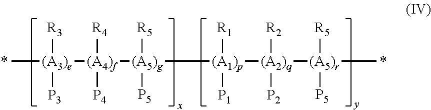
wherein
A₁(R₁)(P₁) is a second block repeating unit, wherein A₁ is the backbone of the repeating unit, R₁ is substituent of the repeating unit selected from the group consisting of hydrogen and C1-C8 alkyl optionally substituted with one or more fluorine atoms, and P₁ is a pendant group comprising a carbohydrate,
A₂(R₂)(P₂) is a second block repeating unit, A₂ is the backbone of the repeating unit, R₂ is substituent of the repeating unit selected from the group consisting of hydrogen and C1-C8 alkyl optionally substituted with one or more fluorine atoms, and P₂ is a pendant group comprising a functional group suitable for associating a therapeutic agent to the block copolymer,
A₃(R₃)(P₃) is a first block repeating unit, wherein A₃ is a backbone of the repeating unit, R₃ is substituent of the repeating unit selected from the group consisting of hydrogen and C1-C8 alkyl optionally substituted with one or more fluorine atoms, and P₃ is a pendant group comprising a carboxylic acid,
A₄(R₄)(P₄) is a first block repeating unit, wherein A₄ is a backbone of the repeating unit, R₄ is substituent of the repeating unit selected from the group consisting of hydrogen and C1-C8 alkyl optionally substituted with one or more fluorine atoms, and P₄ is a pendant group comprising an amine,
A₅(R₅)(P₅) is a repeating unit, wherein A₅ is a backbone of the repeating unit, R₅ is substituent of the repeating unit selected from the group consisting of hydrogen and C1-C8 alkyl optionally substituted with one or more fluorine atoms, and P₅ is a pendant group comprising a neutral group,
e is the mole fraction of the repeating unit in the first block, from 0.0 to 1.0,
f is the mole fraction of the repeating unit in the first block, from 0.0 to 1.0, wherein e+f is at least 0.01,
g is the mole fraction of the repeating unit in the first block, from 0.0 to 0.99,
p is the mole fraction of the repeating unit in the second block, from 0.01 to 0.99,
q is the mole fraction of the repeating unit in the second block, from 0.01 to 0.99,
r is the mole fraction of the repeating unit in the second block, from 0.0 to 0.99,
x is the mole fraction of the first block, from 0.01 to about 0.99, and
y is the mole fraction of the second block, from 0.01 to about 0.99.

31-34. (canceled)
